SEQUENCE LISTING (54 sequences)

SEQ ID NO: 1 - protein, 12 aa, Artificial Sequence
  Synthetic alpha peptide sequence
CTXTXECMPSCN

SEQ ID NO: 2 - protein, 11 aa, Artificial Sequence
  Synthetic beta peptide sequence
LCPTTKCTSXC

SEQ ID NO: 3 - protein, 69 aa, Bacillus halodurans
MTNLLKEWKMPLERTHNNSNPAGDIFQELEDQDILAGVNGACAWYNISCRLGNKGAYCTLTVECMPSCN

SEQ ID NO: 4 - protein, 65 aa, Bacillus halodurans
MVNSKDLRNPEFRKAQGLQFVDEVNEKELSSLAGSGDVHAQTTWPCATVGVSVALCPTTKCTSQC

SEQ ID NO: 5 - protein, 28 aa, Bacillus halodurans
  MOD_RES (18)..(18): Abu
CAWYNIACRLGNKGAYAXLXVEAMPSAN

SEQ ID NO: 6 - protein, 24 aa, Bacillus halodurans
  MOD_RES (1)..(1): Abu
XXWPAAXVGVAVALAPXTKAXXQA

SEQ ID NO: 7 - protein, 74 aa, Bacillus licheniformis
MSKKEMILSWKNPMYRTESSYHPAGNILKELQEEEQHSIAGGTITLSTCAILSKPLGNNGYLCTVTKECMPSCN

SEQ ID NO: 8 - protein, 50 aa, Bacillus licheniformis
VSEEELKALVGGNDVNPETTPATTSSWTCITAGVTVSASLCPTTKCTSRC

SEQ ID NO: 9 - protein, 4 aa, Artificial Sequence
  Synthetic protease cleavage sequence
IEGR

SEQ ID NO: 10 - protein, 69 aa, Artificial Sequence
  Synthetic haloduracin
MTNLLKEWKMPLERTHNNSNPAGDIFQELEDQDILAGIEGRCAWYNISCRLGNKGAYCTLTVECMPSCN

SEQ ID NO: 11 - protein, 65 aa, Artificial Sequence
  Synthetic haloduracin
MVNSKDLRNPEFRKAQGLQFVDEVNEKELSSLAGSGDIEGRTTWPCATVGVSVALCPTTKCTSQC

SEQ ID NO: 12 - DNA, 210 bp, Bacillus halodurans
atgacaaatcttttaaaagaatggaaaatgcctcttgagcgtactcataataattctaatccagcaggggatattttccaagagttagaagaccaagatattctagctggggtgaatggtgcttgcgcatggtacaacatcagctgccgtctaggtaacaaaggtgcttactgcacacttacagttgagtgcatgccttcttgcaactaa

SEQ ID NO: 13 - DNA, 198 bp, Bacillus halodurans
atggtaaattcaaaagatttgcgtaatcctgaattccgcaaagcccaaggtctacaattcgttgacgaggtgaacgagaaggaactttcgtctctagctggttcaggagatgtgcatgcacaaacaacttggccttgcgctacagttggtgtctccgtagccttgtgcccaactacaaagtgtacaagccagtgctaa

SEQ ID NO: 14 - DNA, 225 bp, Bacillus licheniformis
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 15 - DNA, 199 bp, Bacillus licheniformis
tgaaaacaatgaaaaattcagctgcccgtgaagccttcaaaggagccaatcatccggcagggatggtttccgaagaggaattgaaagctttggtaggaggaaatgacgtcaatcctgaaacaactcctgctacaacctcttcttggacttgcatcacagccggtgtaacggtttctgcttcattatgcccaacaactaa

SEQ ID NO: 16 - DNA, 3180 bp, Bacillus halodurans
atgagagaattacaaaatgctctttacttttcagaagtagtgttcgggccaaatttggagaaaatagttggtgagaaacggttgaatttttggctgaagttgatcggggaggatcctgaaaaccttaaggagtttctctcgcgaaaagggaattcttttgaagaacaaacgctcccggaaaaagaagcgatcgtaccgaacagattaggagaagaggcactagaaaaagtaagagaagaattggaatttcttaatacatattcaacgaagcatgtaagaagagtaaaagaactcggtgtccaaattccatttgaaggcattcttctgccgttcatatccatgtatatagagaaatttcaacaacaacaattgaggaaaaaaattggtccaatacatgaagagatttggactcagatagtgcaagatattaccagcaaacttaatgctattctacaccgtacacttatcctggaactgaacgttgcaagagtcacctctcagttgaagggagataccccggaagaacgatttgcttattactcaaaaacctatttagggaaaagggaggtcacccatcgtctttattctgaatatccagttgtcctacgattactatttaccacgattagtcatcatattagctttataacggaaattctcgaacgagttgccaacgatcgagaagcgatagaaacagagttttcaccatgctcaccgatcggtactttggcctcactccacctaaattcaggtgacgctcatcataaacaacgcacggtcactattctagagttttcttcgtcgttaaaacttgtttataaaccgcgttctcttaaggttgacggggtatttaatggcctgcttgcatttttaaatgacagaacaggtgaagtgataaaagatcaatattgtcctaaagtgctacaacgagacggatatggatacgtagaattcgttacccatcaaagctgtcaatcgctagaagaggtatctgatttttatgaaagactcggttcacttatgagcctttcgtatgtcctcaattcatccgactttcactttgaaaatattattgctcatggtccataccctgttttaattgatttagagacgattattcataatacggccgattcatcagaagagacatccacagcgatggatagagcttttcggatgttaaatgattctgtcctttcaacaggtatgctaccttcgtctatctactatcgagatcaaccgaatatgaaagggttaaacgtaggtggtgtcagcaagagcgagggacagaaaacaccatttaaagtcaatcaaattgcaaacagaaatacggatgaaatgagaatagagaaagaccatgtgacgctttctagtcaaaagaacttgccaatttttcaatcggcagcaatggagtctgttcactttctcgaccaaattcaaaagggctttacttcaatgtatcaatggattgaaaaaaacaagcaggaatttaaggaacaggtgcggaagtttgaaggtgtccccgtccgggcggtcctccgttccacgacacgttatacggaacttttaaaaagtagttatcatccagatttactaaggtcagcacttgatcgtgaagttctcttaaacaggttgacggtagattctgtcatgaccccatatttgaaggaaattattcctctcgaagtggaagatttacttaatggggatgttccatatttctatactttgccggaagaacgcgccttatatcaagaggcttccgctatcaattcaacgttttttaccacatctatttttcataaaatcgatcaaaaaatagataaattagggatagaggatcatactcagcaaatgaaaattttgcatatgtcgatgcttgcaagtaacgcgaatcattatgcagatgttgcagaccttgatattcaaaaaggtcatacgatcaaaaatgagcagtatgtagagatggcaaaagatattggagattacttgatggaattatctgtggaaggagaaaatcagggtgaacctgatctttgttggatcagtacggttttagaaggctctagcgaaattatatgggatatttccccggtaggtgaagatctatataatggttcagcaggggtcgccctattttatgcttacttgtttaaaataacaggggagaagcgatatcaagagatcgcctataaagcgttagtccctgttcggagatctgttgctcaattccaacaccaccctaactggagcataggtgcatttaatggagcgagtggttatctttacgcaatgggaacaatcgctgctttatttaatgacgagcgtcttaaacatgaggtaactagaagcatccctcatatagagcctatgatccacgaagataaaatctatgatttcattgggggttcagcaggggcgttgaaagtatttttatctctttccggcctttttgatgagcctaagttcttggaattggcgattgcttgtagtgaacatttaatgaaaaatgcgatcaagactgaccaggggataggatggaaaccaccatgggaggtaactccattaacgggtttttctcatggcgtatcaggcgtaatggcttcgttcattgaactctatcaacagactggagatgaacggctgttatcctatattgatcaatctcttgcgtatgagcgaagtttcttttcagaacaggaggaaaactggctaactccaaacaaagaaacgccagttgttgcgtggtgccatggggcgccagggatacttgtaagtcgcttactattaaagaaatgtggatatcttgatgagaaggtagaaaaagagattgaagtggccttgtctacaacgataagaaaaggtcttggtaacaatcgttccctttgccacggtgactttggccaattggagattttacgatttgcggccgaggttttaggcgattcgtatttacaggaagtagtaaacaatctcagtggtgagttgtacaatcttttcaaaacagaagggtatcagtcgggaacctctagaggaacggaatccgttggtttaatggtggggctgtctggatttggttatggacttttaagtgctgcttatccatctgccgttccttctatactaacgctagacggcgaaattcaaaaataccgtgaaccgcatgaggcgaatcattaa

SEQ ID NO: 17 - DNA, 2973 bp, Bacillus halodurans
atgaaaactcctctaacaagtgagcatcctagcgtaccaacaacacttccgcacaccaacgatacagattggctagaacaacttcatgacatcttatcgatccctgtcaccgaagagatccagaaatacttccatgcagaaaatgatttgttttcttttttctatactccctttcttcaatttacatatcaatcaatgtccgactattttatgactttcaaaacggacatggcgctcatagaacggcaatcacttctacaatcaaccttaacagctgttcaccatcgcctctttcacttaactcatcgtacgttaatttcagaaatgcacattgataaattaactgtaggcttaaacggatcaacaccacacgaacgttatatggattttaaccataaatttaataaaacttcaaaatcaaaaaatttatttaatatatatccaattctaggtaaacttgtagtaaatgagacgttgcgcaccatcaattttgtaaaaaagatcattcagcattatatgaaagactacctattgttgagtgatttttttaaagagaaagacttacgactaacgaaccttcagctcggcgtcggggatactcatgtaaatggtcaatgtgtgaccatccttacatttgcctctggccaaaaagtagtctacaagccccgttccctgtcaattgataaacaatttggtgagtttattgagtgggttaattccaaaggatttcaaccatcattacgcattcctatagccattgatcgacaaacatacggctggtatgaatttatcccacatcaagaggcaacatcggaggatgagatcgagcgctattattcaagaatcggaggatatctggctatcgcttatcttttcggagcgacagatcttcatttagacaatttaatcgcctgcggagaacacccgatgttaattgatttagaaacattattcaccaatgatttggattgttacgacagcgcatttccattccctgctcttgctagggagctaacacaatctgtctttgggaccttgatgcttccaattacaattgctagtggcaaattacttgatatcgatctaagtgcagtcggcgggggaaaaggcgttcaatctgaaaagattaaaacatgggttatcgtcaatcaaaaaacggatgagatgaagttagttgaacaaccttacgtaaccgaatctagccaaaataaacctacagttaacgggaaagaagcgaatatcgggaattacatcccccatgttacagatggttttcggaaaatgtatcgactctttctcaacgaaatcgatgaactaatggaccacaacgggcctatcttcgcttttgaatcatgtcaaattcgccatgtgtttcgggccactcatgtttatgctaaatttttggaggctagcactcaccccgactatttgcaggaaccgactaggcggaataagctgtttgaatcattctggaacattacctcgttaatggctccatttaagaagatcgtgccacatgaaatagcagaacttgaaaaccatgatatcccttattttgtccttacttgtggcggaaccatcgttaaggatggatatggaagagacatcgctgacctctttcaatcctcttgtattgaacgagtcacccatcgactacaacagttagggagtgaagatgaagcaagacaaattcgctatatcaagagctctcttgctacccttactaatggcgactggacaccttctcacgaaaaaacaccaatgtcacctgcttctgctgaccgagaagatggatactttttgcgagaagcccaagccataggagatgacattttagcgcaactcatttgggaagacgatcggcatgccgcttatttaattggtgtaagtgttggaatgaatgaagccgtaaccgtctcccctctcacaccagggatttatgatggtacattaggaattgttttgtttttcgatcaacttgctcaacaaacaggagaaacacactaccggcatgctgctgatgctttgttggaaggaatgtttaaacagctaaagcctgagctaatgccatcttctgcttatttcggtttagggtcactgttctatggccttatggtgcttggactccaacggtctgattcacacatcattcaaaaggcatatgaatacttgaagcatttggaagaatgtgttcagcacgaagaaacaccggactttgtcagtggtttatctggagttctttatatgctgacaaaaatctatcaactgacaaacgagcctagagtttttgaggtagcaaagacgaccgcaagccgattgtcagtgctattagattctaaacagccagatacggttcttacggggcttagccatggagccgcaggatttgcgttagctttactcacttacggaacggctgcgaatgatgaacagcttttgaaacaagggcactcgtaccttgtctatgaacggaaccgatttaacaaacaggaaaacaattgggtggatttaaggaagggaaacgcctatcaaaccttttggtgccatggtgctcctgggattgggatcagccgtcttttactagcacagttctacgatgacgaacttcttcacgaagaattaaatgctgcattaaacaaaaccatatcagacgggttcgggcataatcatagtctctgtcacggtgatttcggaaatttagatcttttactgttgtatgcccagtatacgaacaatcccgagccaaaagagctagcaagaaaattagcgatttcttctatcgaccaagcgcacacatatggctggaaattgggcttaaatcatagcgatcaattgcaaggaatgatgttaggggtcacaggtattggctatcaattgcttcgacatattaaccctaccgttccatcgattttagcactggagcttccctcttctacattaaccgaaaaagagttgagaattcatgacagataa

SEQ ID NO: 18 - DNA, 3159 bp, Bacillus licheniformis
atgaatgaaaaatccgccggatatcacgaacggcttcccgtcgcccaaactcaatccccgctcgtaaacgataagataaagtattggcgttcccttttcggcgatgatgataaatggctcaataaagcagtttcattattaagccatgaccctttgtcctccatcgcacaatcctcggtatcccagtcagtcgggctgaaagacagccgtcgcggcccatggcagaagatgcaaaagcggatctttgaaacgcccttttcctacaaggattctgctctgcaagattcagaattgctgttcgactccctgctgacccgttttgcgtctgcagcacaagatgctttggaggaacaaaatatcatactttctcctcctctttgccggcaggtgctgacacatttaaaacagacgcttcttcaaattgcccatcaaacattaatactggaactaaacattttaaggcttgaagatcaattgaagggcgacacccccgaaatgcgctatcttgatttcaatgataactttttagtcaatccaggatacctgcggaccctgttcaacgagtatcccgtattgctgcgccttctgtgcacaaaaaccgattactgggttcaaaacttttctgaactgtggaagaggctgaggcaggaccgcgaacagctgcaggctgcatttcatattgccggcgatcctgtccatattgagcttggggtgggagactcgcacaataaaggaaagatggcagccatccttacatattccgatggaaaaaagattgtctataaaccgagaagccatgatgttgacgacgcatttcaacttcttctatcatggatcaatgaccgaaattcaggcagccctttaaaaactttgagattaatcaataaaaaacggtacggatggtccgagtttattcctcacgaaacgtgccatacgaaaaaagaactggaaggctactatacacgcctcggcaaacttttggccgttttatacagcatcgatgccgttgactttcaccacgaaaacattatcgcctccggcgagcatcctgttttaatcgatcttgaatcaatttttcatcaatataaaaaacgagacgaacccggctcgaccgccgttgacaaagcaaactacattctttccagatccgtacggtctaccggaatcctgccgttcaacctttacttcggaaggaaaaaccgggataaagttgtggacatcagcggaatgggggggcaggaagctcaggaatcaccgtttcaggcgcttcaaatcaaaggatttttccgcgatgacattcgcctggagcatgaccgatttgaaatcggcgaggcgaaaaatctgccgactttagatcaccagcatgtccctgtcgcagattatcttcattgtatcatcgaaggattttcagcagtataccgtctgatttctgatcatggcgaaagctacctggctacgattgaacattttaaaaactgcaccgttcgaaatattttgaagccgacagcgcactacgcctctcttttgaataaaagctaccaccctgattttctcagggatgcggtagaccgtgaagtgtttttatgccgggtggaaaagtttgaagatgcagacacagatattgcagcggcaaaaacagagctgaaagagctcattcggggagacatcccctattttctgtcgaagccttcagatacctatttgctcaatggcgaagaagaaccgattgccgcttattttgaaacgccgtccttcacaagagtaattaagaagatctcatcattttcagaccaggacttaaaggaacaagcgaatgtcatacgcatgtcgattctggctgcatataacgcgagacatgaaaaagacgcaatcgatatagaccaaaatcacccgagtcctagatcaggcgccttgcagccgctcgccatcgctgagaaagcggctgacgatttggctgaaaagcgaattgaaggcaatgatggaaaggacgtcacttggatcagtacagttattgaaggcgtcgaagaaatctcttggacgatctcccctgtcagtcttgatttatataatggcaatgcaggcatcggactttttatgagctatctgagccgcttcgcaaaacggccggagacttactcgcatataaccgagcagtgtgtatttgcgattcagcgagcgttgaatgaactgaaggaaaaagaagaattcctgaagtacgccgactctggggcattcacgggggtttccggctatctgtattttctgcagcatgcgggaacggttcagaaaaaaaacgaatggatcgaactcatacatgaagctctgccagtccttgaagctgtcatcgaacaagacgaaaactgcgatatcatcagcggttctgccggtgctctaatggttctgatgtcattgtatgaacaactggatgacccggtttttctaaagctcgccgaaaagtgcgccggccatttgcttcagcataaaacaaatattgaaaacggagcggcctggaaagatcctcatacacaaaactattacacaggatttgcccacggcacttccggcatcgccgcagctttatcccgattcaataaagtgtttgattcgcaatcactgaaaaaaatcatttcgcaatgcctggcatttgaaaagcagctgtacatcgcttccgaaaaaaattggggatcaaaaggaagagaacaactgtcagttgcatggtgccatggcgctgccggcatattgttgtcgagaagcatcctccgagaaaacggagtcaatgatcccgggctgcataccgacatcttgaacgctcttgaaacaactgttaagcatgggctcggcaataaccgctcattctgtcacggcgatttcggccaactcgaaatcctaagagggttcagggaagaattcagcgaactgaacaccattatacagaatacggaagatcggctgttgacatattttcaagaaaatccattcagtaaaggggtatcacgaggtgtggattcagccgggctcatgcttggtttaagcggagtcggctacggcatgctgcaatgccaatatggagaagaactgccggaactgcttcagctcagtccgcctcaagcgcttatcaaaaagaacagcaaagcttttaaaagagaaaacgtgttttaa

SEQ ID NO: 19 - DNA, 3081 bp, Bacillus licheniformis
atgagcatgaaagaattcgaaatttatctgtataaagctctttacagcaatgaacgaggcggtcaaggtcaagaacatccgtccggcttttttccggaaaacggaaaaactccgtctcgtcctacggattttcacctttcttctgtccaacattcacccaatgagcctgtgcagctgcaaggcaaaatgccggaatgggctgcctgtttgtctgaaattatgaaatacaaccctaaagcggtttccgaattaaaacacccgcttccccacatgtcatttgtcaccttcttggttccttttcttttatttgcacaagaacggatgtcgaaagctttttctgaatttgagaagcaggaaggcggtctatccggcataatcgacgctgccggctatcaagacggcatcatgtctgaacttcaccaatgccttgataagctggcgacgagaacacttatcacagagctgaatgtagcccgggaagacggccggctaaagggggcgtcaccggaagagcgatatgtttactttgttgaacaatacatttccgatcctgaaatttaccgggaatttttcgagctttaccctgtgcttggcaggctgatggctgagaaggttctcagggtgctcgagattcatgaagaaattattgggagatttttaagcgaccgcagcctgattgcgaaaaaatttaatatcgcttcccccgaattggttggatttgaaggggatttgggagattcccacaaaaacgggcagagtgtcaaagtgctggtgttaaacaacggaaagctcgtgtataaaccgcggtccttgtcaattgacgaacattacagggagctgctgaactggctgaacggacggggaatgaagtacagcctccgtgctgcggaagtgcttgacaggggaaattacggctggcaggaatttgtaaagcatgaaggctgttcttcagaagaagaactggaaagattttatttccggcagggcggacatttggcgatattgtacggattgcgctccgtcgattttcataatgaaaatatcatcgcctcaggcgaacaccccatcctgattgatctggagactctttttgacaaccatgtcagcattttcgctcaaaatcaaaacctccatgtcaccgcattggagctgaagcattccgtgctgtcttcgatgatgcttccggtcaaattcaaacatgatgaagtgctcgattttgatttaagcgggatcggcggcaaaggcggccagcagtcaaagaaagcgaagggctacgccgtcctgaattacggtgaggacaggatgtctttaaaagaaacatcgctgaccaccgaggaaaaattgaatgcgcccaaactaaatggacgtccggtgtccgccgttttctatacggactttatcgtggaaggatttaaaaatgcttatgccattatgatgaaacataaagaagaactggcaggaccctcggggtttttgaatctgtttaagcacgacgaagtccgccacgtcttccggccgacacatgtgtacggcaagtttctcgaagcgagcacccaccctgactacttgaccgccggagataaacgagagcaattatttgattatatgtggatgctcgccaaacagtcggaaaaggcaaacgtgtttattccggacgaaattgtcgatctgctgcttcatgatattccctactttactttttatgctgggggcacttccctgctcaattcaagaggggaagaatcggaaggtttttatgaaacatcaagtattgatttggcaaagaaaaaaattcaatctttctcggaaaaggatttgaatcatcagctccgctatatttctttatcaatggcgacgttgattgaaaatgtctgggaccatgcagaaagcgggcttggacagaaggaaacggtcgctgatctcggaaaagaggtcaagcatatagctgatgatttgctgcagaaggcgatctattcagagcgcggtgaaggtcctttctggatcagcaataatgccggagacgaaaaaatggtgtttttgtcgccgcttcctatggggctttacgacggaatggcagggctggcaatattttttgcacaagcaggcaaggtactgaacgagcaggtatatacggatacggcaagatcaatgatagaagaaattcaaaaggaagaaagttattgggttcaaaatgggaattcccattctgcttttttcggcacaggctcattcatttacctgtattcctatcttggcagtctatgggaagacgattccttattggaaagggcgttgaacctcattccccgagttttggatcagccgaatcaaacacaaaacccggattttatcgcaggggattcaggattgctgacagtgcttgttaatctgtacgaaatcaagcagcacccagcagtattggactctataagacaggtactgagcagattgaatgatcgaattggccgcttacttgattcaatcgagcaggatgccgtttcgttgacgggattttcacacggcttgacggggatcgcattttctatcgcaaaggcggcgaaggtgatacacgatgacagctgcaaagagcttgtcctaaagcttgtcgaagaagaggaccgctattttcaaaaggatcatctaaactggctagatttacgaaatgattcgcatacgctgtccccaagctactggtgtcatggagctcccgggattttgctggggagagcgcacattcaggcttttattcctgaattgactacccggactttaaagcttcaagaagcgcttcaaagttctttaaatctagcagactgtcaaaatcattcgctgtgccacggtttaattgggaatttgaacattctgctggatatcaaaaggctgaaccgggaacttcatgtccctgatgatatattttgcatttataaaacgaaaaaccggggatggaaaacgggtttgcattccgatgtggaatcgcttggcatgtttgtcgggacggcaggaatagcctacgggcttttgcggctcctcgatgaatctgttccatccgtattaactctcgatattccgacgggcaggtga

SEQ ID NO: 20 - protein, 1059 aa, Bacillus halodurans
MRELQNALYFSEVVFGPNLEKIVGEKRLNFWLKLIGEDPENLKEFLSRKGNSFEEQTLPEKEAIVPNRLGEEALEKVREELEFLNTYSTKHVRRVKELGVQIPFEGILLPFISMYIEKFQQQQLRKKIGPIHEEIWTQIVQDITSKLNAILHRTLILELNVARVTSQLKGDTPEERFAYYSKTYLGKREVTHRLYSEYPVVLRLLFTTISHHISFITEILERVANDREAIETEFSPCSPIGTLASLHLNSGDAHHKQRTVTILEFSSSLKLVYKPRSLKVDGVFNGLLAFLNDRTGEVIKDQYCPKVLQRDGYGYVEFVTHQSCQSLEEVSDFYERLGSLMSLSYVLNSSDFHFENIIAHGPYPVLIDLETIIHNTADSSEETSTAMDRAFRMLNDSVLSTGMLPSSIYYRDQPNMKGLNVGGVSKSEGQKTPFKVNQIANRNTDEMRIEKDHVTLSSQKNLPIFQSAAMESVHFLDQIQKGFTSMYQWIEKNKQEFKEQVRKFEGVPVRAVLRSTTRYTELLKSSYHPDLLRSALDREVLLNRLTVDSVMTPYLKEIIPLEVEDLLNGDVPYFYTLPEERALYQEASAINSTFFTTSIFHKIDQKIDKLGIEDHTQQMKILHMSMLASNANHYADVADLDIQKGHTIKNEQYVEMAKDIGDYLMELSVEGENQGEPDLCWISTVLEGSSEIIWDISPVGEDLYNGSAGVALFYAYLFKITGEKRYQEIAYKALVPVRRSVAQFQHHPNWSIGAFNGASGYLYAMGTIAALFNDERLKHEVTRSIPHIEPMIHEDKIYDFIGGSAGALKVFLSLSGLFDEPKFLELAIACSEHLMKNAIKTDQGIGWKPPWEVTPLTGFSHGVSGVMASFIELYQQTGDERLLSYIDQSLAYERSFFSEQEENWLTPNKETPVVAWCHGAPGILVSRLLLKKCGYLDEKVEKEIEVALSTTIRKGLGNNRSLCHGDFGQLEILRFAAEVLGDSYLQEVVNNLSGELYNLFKTEGYQSGTSRGTESVGLMVGLSGFGYGLLSAAYPSAVPSILTLDGEIQKYREPHEANH

SEQ ID NO: 21 - protein, 988 aa, Bacillus halodurans
TPLTSEHPSVPTTLPHTNDTDWLEQLHDILSIPVTEEIQKYFHAENDLFSFFYTPFLQFTYQSMSDYFMTFKTDMALIERQSLLQSTLTAVHHRLFHLTHRTLISEMHIDKLTVGLNGSTPHERYMDFNHKFNKTSKSKNLFNIYPILGKLVVNETLRTINFVKKIIQHYMKDYLLLSDFFKEKDLRLTNLQLGVGDTHVNGQCVTILTFASGQKVVYKPRSLSIDKQFGEFIEWVNSKGFQPSLRIPIAIDRQTYGWYEFIPHQEATSEDEIERYYSRIGGYLAIAYLFGATDLHLDNLIACGEHPMLIDLETLFTNDLDCYDSAFPFPALARELTQSVFGTLMLPITIASGKLLDIDLSAVGGGKGVQSEKIKTWVIVNQKTDEMKLVEQPYVTESSQNKPTVNGKEANIGNYIPHVTDGFRKMYRLFLNEIDELMDHNGPIFAFESCQIRHVFRATHVYAKFLEASTHPDYLQEPTRRNKLFESFWNITSLMAPFKKIVPHEIAELENHDIPYFVLTCGGTIVKDGYGRDIADLFQSSCIERVTHRLQQLGSEDEARQIRYIKSSLATLTNGDWTPSHEKTPMSPASADREDGYFLREAQAIGDDILAQLIWEDDRHAAYLIGVSVGMNEAVTVSPLTPGIYDGTLGIVLFFDQLAQQTGETHYRHAADALLEGMFKQLKPELMPSSAYFGLGSLFYGLMVLGLQRSDSHIIQKAYEYLKHLEECVQHEETPDFVSGLSGVLYMLTKIYQLTNEPRVFEVAKTTASRLSVLLDSKQPDTVLTGLSHGAAGFALALLTYGTAANDEQLLKQGHSYLVYERNRFNKQENNWVDLRKGNAYQTFWCHGAPGIGISRLLLAQFYDDELLHEELNAALNKTISDGFGHNHSLCHGDFGNLDLLLLYAQYTNNPEPKELARKLAISSIDQAHTYGWKLGLNHSDQLQGMMLGVTGIGYQLLRHINPTVPSILALELPSSTLTEKELRIHDR

SEQ ID NO: 22 - protein, 1052 aa, Bacillus licheniformis
MNEKSAGYHERLPVAQTQSPLVNDKIKYWRSLFGDDDKWLNKAVSLLSHDPLSSIAQSSVSQSVGLKDSRRGPWQKMQKRIFETPFSYKDSALQDSELLFDSLLTRFASAAQDALEEQNIILSPPLCRQVLTHLKQTLLQIAHQTLILELNILRLEDQLKGDTPEMRYLDFNDNFLVNPGYLRTLFNEYPVLLRLLCTKTDYWVQNFSELWKRLRQDREQLQAAFHIAGDPVHIELGVGDSHNKGKMAAILTYSDGKKIVYKPRSHDVDDAFQLLLSWINDRNSGSPLKTLRLINKKRYGWSEFIPHETCHTKKELEGYYTRLGKLLAVLYSIDAVDFHHENIIASGEHPVLIDLESIFHQYKKRDEPGSTAVDKANYILSRSVRSTGILPFNLYFGRKNRDKVVDISGMGGQEAQESPFQALQIKGFFRDDIRLEHDRFEIGEAKNLPTLDHQHVPVADYLHCIIEGFSAVYRLISDHGESYLATIEHFKNCTVRNILKPTAHYASLLNKSYHPDFLRDAVDREVFLCRVEKFEDADTDIAAAKTELKELIRGDIPYFLSKPSDTYLLNGEEEPIAAYFETPSFTRVIKKISSFSDQDLKEQANVIRMSILAAYNARHEKDAIDIDQNHPSPRSGALQPLAIAEKAADDLAEKRIEGNDGKDVTWISTVIEGVEEISWTISPVSLDLYNGNAGIGLFMSYLSRFAKRPETYSHITEQCVFAIQRALNELKEKEEFLKYADSGAFTGVSGYLYFLQHAGTVQKKNEWIELIHEALPVLEAVIEQDENCDIISGSAGALMVLMSLYEQLDDPVFLKLAEKCAGHLLQHKTNIENGAAWKDPHTQNYYTGFAHGTSGIAAALSRFNKVFDSQSLKKIISQCLAFEKQLYIASEKNWGSKGREQLSVAWCHGAAGILLSRSILRENGVNDPGLHTDILNALETTVKHGLGNNRSFCHGDFGQLEILRGFREEFSELNTIIQNTEDRLLTYFQENPFSKGVSRGVDSAGLMLGLSGVGYGMLQCQYGEELPELLQLSPPQALIKKNSKAFKRENVF

SEQ ID NO: 23 - protein, 1026 aa, Bacillus licheniformis
MSMKEFEIYLYKALYSNERGGQGQEHPSGFFPENGKTPSRPTDFHLSSVQHSPNEPVQLQGKMPEWAACLSEIMKYNPKAVSELKHPLPHMSFVTFLVPFLLFAQERMSKAFSEFEKQEGGLSGIIDAAGYQDGIMSELHQCLDKLATRTLITELNVAREDGRLKGASPEERYVYFVEQYISDPEIYREFFELYPVLGRLMAEKVLRVLEIHEEIIGRFLSDRSLIAKKFNIASPELVGFEGDLGDSHKNGQSVKVLVLNNGKLVYKPRSLSIDEHYRELLNWLNGRGMKYSLRAAEVLDRGNYGWQEFVKHEGCSSEEELERFYFRQGGHLAILYGLRSVDFHNENIIASGEHPILIDLETLFDNHVSIFAQNQNLHVTALELKHSVLSSMMLPVKFKHDEVLDFDLSGIGGKGGQQSKKAKGYAVLNYGEDRMSLKETSLTTEEKLNAPKLNGRPVSAVFYTDFIVEGFKNAYAIMMKHKEELAGPSGFLNLFKHDEVRHVFRPTHVYGKFLEASTHPDYLTAGDKREQLFDYMWMLAKQSEKANVFIPDEIVDLLLHDIPYFTFYAGGTSLLNSRGEESEGFYETSSIDLAKKKIQSFSEKDLNHQLRYISLSMATLIENVWDHAESGLGQKETVADLGKEVKHIADDLLQKAIYSERGEGPFWISNNAGDEKMVFLSPLPMGLYDGMAGLAIFFAQAGKVLNEQVYTDTARSMIEEIQKEESYWVQNGNSHSAFFGTGSFIYLYSYLGSLWEDDSLLERALNLIPRVLDQPNQTQNPDFIAGDSGLLTVLVNLYEIKQHPAVLDSIRQVLSRLNDRIGRLLDSIEQDAVSLTGFSHGLTGIAFSIAKAAKVIHDDSCKELVLKLVEEEDRYFQKDHLNWLDLRNDSHTLSPSYWCHGAPGILLGRAHIQAFIPELTTRTLKLQEALQSSLNLADCQNHSLCHGLIGNLNILLDIKRLNRELHVPDDIFCIYKTKNRGWKTGLHSDVESLGMFVGTAGIAYGLLRLLDESVPSVLTLDIPTGR

SEQ ID NO: 24 - DNA, 21 bp, Artificial sequence
  Synthetic oligonucleotide
gcgccgcatatgacaaatctt

SEQ ID NO: 25 - DNA, 21 bp, Artificial sequence
  Synthetic oligonucleotide
aggctcgagttagttgcaaga

SEQ ID NO: 26 - DNA, 21 bp, Artificial sequence
  Synthetic oligonucleotide
gcgccgcatatggtaaattca

SEQ ID NO: 27 - DNA, 21 bp, Artificial sequence
  Synthetic oligonucleotide
aaactcgagttagcactggct

SEQ ID NO: 28 - DNA, 21 bp, Artificial sequence
  Synthetic oligonucleotide
gccgctagcatgagagaatta

SEQ ID NO: 29 - DNA, 21 bp, Artificial sequence
  Synthetic oligonucleotide
cgtctcgagttaatgattcgc

SEQ ID NO: 30 - DNA, 36 bp, Artificial sequence
  Synthetic oligonucleotide
gggtatccgctcgagatgaaaactcctctaacaagt

SEQ ID NO: 31 - DNA, 36 bp, Artificial sequence
  Synthetic oligonucleotide
tataaacgcggatccttatctgtcatgaattctcaa

SEQ ID NO: 32 - DNA, 36 bp, Artificial sequence
  Synthetic oligonucleotide
attctagctgggattgaaggtcgttgcgcatggtac

SEQ ID NO: 33 - DNA, 21 bp, Artificial sequence
  Synthetic oligonucleotide
aggctcgagttagttgcaaga

SEQ ID NO: 34 - DNA, 36 bp, Artificial sequence
  Synthetic oligonucleotide
gcttcaggagatattgaaggtcgtacaacttggcct

SEQ ID NO: 35 - DNA, 21 bp, Artificial sequence
  Synthetic oligonucleotide
aaactcgagttagcactggct

SEQ ID NO: 36 - protein, 4 aa, Artificial sequence
  Synthetic peptide
VNGA

SEQ ID NO: 37 - protein, 4 aa, Artificial sequence
  Synthetic peptide
VHAQ

SEQ ID NO: 38 - protein, 41 aa, Bacillus halodurans
MTNLLKEWKMPLERTHNNSNPAGDIFQELEDQDILAGVNGA

SEQ ID NO: 39 - protein, 35 aa, Bacillus halodurans
MVNSKDLRNPEFRKAQGLQFVDEVNEKELSSLAGS

SEQ ID NO: 40 - protein, 4 aa, Artificial sequence
  Synthetic peptide
AWPS

SEQ ID NO: 41 - protein, 59 aa, Lactobacillus plantarum
MKISKIEAQARKDFFKKIDTNSNLLNVNGAKCKWWNISCDLGNNGHVCTLSHECQVSCN

SEQ ID NO: 42 - protein, 62 aa, Bacillus licheniformis
MKSSFLEKDIEEQVTWFEEVSEQEFDDDIFGACSTNTFSLSDYWGNKGNWCTATHECMSWCK

SEQ ID NO: 43 - protein, 59 aa, Lactococcus lactis
MNKNEIETQPVTWLEEVSDQNFDEDVFGACSTNTFSLSDYWGNNGAWCTLTHECMAWCK

SEQ ID NO: 44 - protein, 61 aa, Streptococcus ratti
MKEIQKAGLQEELSILMDDANNLEQLTAGIGTTVVNSTFSIVLGNKGYICTVTVECMRNCQ

SEQ ID NO: 45 - protein, 62 aa, Streptococcus mutans
MKEIQKAGLQEELSILMDDANNLEQLTAGIGTTVVNSTFSIVLGNKGYICTVTVECMRNCSK

SEQ ID NO: 46 - protein, 67 aa, Lactobacillus plantarum
MTKTSRRKNAIANYLEPVDEKSINESFGAGDPEARSGIPCTIGAAVAASIAVCPTTKCSKRCGKRKK

SEQ ID NO: 47 - protein, 62 aa, Streptococcus ratti
MKSNLLKINNVTEVEKDMVTLIKDEDMELAGGSTPACAIGVVGITVAVTGISTACTSRCINK

SEQ ID NO: 48 - protein, 61 aa, Streptococcus mutans
MKSNLLKINNVTEMEKNMVTLIKDEDMLAGGSTPACAIGVVGITVAVTGISTACTSRCINK

SEQ ID NO: 49 - protein, 65 aa, Lactococcus lactis
MKEKNMKKNDTIELQLGKYLEDDMIELAEGDESHGGTTPATPAISILSAYISTNTCPTTKCTRAC

SEQ ID NO: 50 - protein, 67 aa, Bacillus licheniformis
MKNELGKFLEENELELGKFSESDMLEITDDEVYAAGTPLALLGGAATGVIGYISNQTCPTTACTRAC

SEQ ID NO: 51 - protein, 63 aa, Enterococcus faecalis
MLNKENQENYYSNKLELVGPSFEELSLEEMEAIQGSGDVQAETTPACFTIGLGVGALFSAKFC

SEQ ID NO: 52 - protein, 68 aa, Enterococcus faecalis
MENLSVVPSFEELSVEEMEAIQGSGDVQAETTPVCAVAATAAASSAACGWVGGGIFTGVTVVVSLKHC

SEQ ID NO: 53 - protein, 29 aa, Lactobacillus plantarum
  MOD_RES (19)..(19): Abu
KCKWWNIACDLGNNGHVAXLAHEAQVSAN

SEQ ID NO: 54 - protein, 28 aa, Lactococcus lactis
  MOD_RES (1)..(1): Dehydrobutyrine
XPAXPAIXILXAYIATNTAPXTKAXRAA

(19) United States
(12) Patent Application Publication
(10) Pub. No.: US 2008/0026999 A1
(43) Pub. Date: Jan. 31, 2008

(54) Two-component bacillus lantibiotic and methods for producing and using the same

(76) Inventor: Wilfred A. van der Donk, Champaign, IL (US)

Correspondence Address: Jane Massey Licata;Licata & Tyrrell P.C., 66 E. Main Street, Marlton, NJ, 08053, US

(21) Appl. No.: 11/768,406

(22) Filed: Jun. 26, 2007

Related U.S. Application Data
(60) Provisional application No. 60/820,646, filed on Jul. 28, 2006.

(51) Int. Cl. A61K 38/00; A61P 43/00; C07K 14/00; C12N 1/20; C12N 15/00; C12N 15/11; C12N 5/06; C12N 9/00; C12P 21/06

(52) U.S. Cl. 514/12; 435/183; 435/245; 435/320.1; 435/325; 435/68.1; 530/324; 536/23.2; 536/23.7

(57) ABSTRACT

The present invention relates to two-component lantibiotics isolated from Bacillus sp. Methods for producing said lantibiotics are provided, wherein dehydration and cyclization of the peptides is carried out by two substrate-specific modifying enzymes. Given the antimicrobial activity of the instant lantibiotics, methods for preventing or treating bacterial infections are also provided.

### INTRODUCTION

This application claims benefit of priority from U.S. Provisional Patent Application Ser. No. 60/820,646 filed Jul. 28, 2006, the content of which is incorporated herein by reference in its entirety.

This invention was made in the course of research sponsored by the National Institutes of Health, grant number GM 58822. The U.S. government may have certain rights in this invention.

### BACKGROUND OF THE INVENTION

Antimicrobial peptides are produced by a wide variety of organisms including bacteria, insects, and mammals (Hancock (1998) Expert Opin. Investig. Drugs 7:167-74; Jack & Jung (2000) Curr. Opin. Chem. Biol. 4:310-7; Toke (2005) Biopolymers 80:717-735). Due to the rapid spread of multiple-drug resistant bacterial strains, antimicrobial peptides are currently being investigated as a potential new source of antibiotics to treat infections. Antimicrobial peptides have a high degree of structural and chemical diversity, exhibit rapid bactericidal action, and typically display a broad spectrum of activity. The lantibiotic group of bacterial-derived antimicrobial peptides possesses high antibacterial activity against Gram positive bacteria including drug resistant strains (Delves-Broughton, et al. (1996) Antonie vanLeeuwenhoek 69:193-202; Kruszewska, et al. (2004) J. Antimicrob. Chemother. 54:648-53; Brumfitt, et al. (2002) J. Antimicrob. Chemother. 50:731-4; Galvin, et al. (1999) Lett. Appl. Microbiol. 28:355-8; Goldstein, et al. (1998) J. Antimicrob. Chemother. 42:277-8; Cotter, et al. (2005) Nat. Rev. Microbiol. 3:777-88). Over 45 members have been identified in the lantibiotic family (Chatterjee, et al. (2005) Chem. Rev. 105:633-84). The most studied lantibiotic, nisin, is produced by Lactococcus lactis and has been used world-wide in food preservation for over 40 years (Delves-Broughton, et al. (1996) supra; Hurst (1981) Adv. Appl. Microbiol. 27:85-123; Rayman, et al. (1981) Appl. Environ. Microbiol. 41:375-80). Lantibiotics share the presence of lanthionine (Lan) and/or methyllanthionine (MeLan) residues, and also typically the unsaturated amino acids dehydroalanine (Dha) and dehydrobutyrine (Dhb) . These structural motifs are the basis for their biological activity as well as their family name (Schnell, et al. (1988) Nature 333:276-278).

Lantibiotics are ribosomally synthesized as precursor peptides (prepeptides) that are subjected to post-translational modifications to produce the active, mature compounds (Cotter, et al. (2005) Nat. Rev. Microbiol. 3:777-88; Chatterjee, et al. (2005) Chem. Rev. 105:633-84). The prepeptide contains an amino-terminal leader sequence that does not undergo post-translational modification. The role of this leader sequence appears to be required for modification of the structural region and must be removed by proteolysis in the final step to produce the mature lantibiotic (Schnell, et al. (1988) Nature 333:276-278; van der Meer, et al. (1994) J. Biol. Chem. 269:3555-62; Xie, et al. (2004) Science 303:679-81; Li, et al. (2006) Science 5766:1464-7). The dehydro amino acids (Dha and Dhb) found in lantibiotics are introduced via the dehydration of serine and threonine residues located in the carboxy-terminal structural region of the prepeptide. Lanthionine (Lan) and methyllanthionine (MeLan) rings can then be generated by intramolecular conjugate additions of cysteine residues to these α,β-unsaturated amino acids.

A growing class of two-component lantibiotic systems utilizes two peptides that are each post-translationally modified to an active form and act in synergy to provide antibacterial activity (Garneau, et al. (2002) Biochimie 84:577-92). Dehydration and cyclization of the prepeptides to form lanthionine bridges in these systems is likely performed by bifunctional LanM proteins. In most cases the sequence similarity of the two peptides is rather low (˜25%), and so two different enzymes are thought to be employed for the post-translational modification of each peptide (McAuliffe, et al. (2000) Microbiology 146:2147-54). The exception is cytolysin, a two-component lantibiotic that is processed by a single LanM enzyme (Cox, et al. (2005) Curr. Protein Pept. Sci. 6:77-84). In this case, the sequence homology of the two peptide substrates is much higher at ˜90%. Other post-translational modifications of the peptides in two-component systems can include the conversion of L-Ser to D-Ala (Skaugen, et al. (1994) J. Biol. Chem. 269:27183-27185; Cotter, et al. (2005) Proc. Natl. Acad. Sci. USA 102:18584-9) and formation of amino-terminal α-keto amides from the deamination of dehydro residues (Martin, et al. (2004) Biochemistry 43:3049-3056).

The best-studied two-component lantibiotic, lacticin 3147, is composed of the modified peptides LtnA1 and LtnA2, and is produced by Lactococcus lactis (Ryan, et al. (1999) J. Biol. Chem. 274:37544-50). Since the designation LtnA1 and LtnA2 also refers to the unmodified prepeptides, the designations Ltn1 and Ltn2 are used herein for the mature, active components. The post-translational modification of each prepeptide is believed to be catalyzed by two separate bifunctional enzymes, LtnM1 and LtnM2, based on genetic data in which deletion of either LanM gene results in abrogation of bioactive material (McAuliffe, et al. (2000) supra). To date, in vitro activity of LtnM1 or LtnM2 has not been demonstrated. The Ltn1 and Ltn2 peptides act in synergy in a 1:1 ratio to produce nanomolar antibacterial activity (Morgan, et al. (2005) Antimicrob. Agents Chemother. 49:2606-11). A study on the mode of action of lacticin 3147 demonstrated that Ltn1 binds to the peptidoglycan precursor lipid II (Wiedemann, et al. (Jun. 12, 2006) Mol. Microbiol.), a result that was anticipated because of the structural similarity between Ltn1 and mersacidin, which also disrupts cell wall biosynthesis by binding to lipid II (Brötz, et al. (1998) Mol. Microbiol. 30:317-327). In order for lacticin 3147 to substantially inhibit cell wall biosynthesis and form small pores in the cell membrane, however, Ltn2 is also necessary, leading to a proposed model in which the lipid II:Ltn1 complex recruits Ltn2 to form a high affinity complex (Wiedemann, et al. (Jun. 12, 2006) supra). Structural characterization of the modified peptides has indicated that Ltn1 adopts a globular conformation similar to mersacidin, while Ltn2 has a more elongated structure that is α-helical in nature (Martin, et al. (2004) supra).

The mechanisms governing substrate recognition and specificity in two-component lantibiotic systems that utilize two modification enzymes are of great interest since it is believed that each LanM protein is required to discriminate between the two prepeptides present in the cell. Needed in the art is a method for in vitro reconstitution of a two-component lantibiotic biosynthetic system to provide definitive support for the roles of the proteins involved and demonstrate recognition and specificity. Such a system could be used to develop novel lantibiotics based on designing peptide sequences that can be site-specifically modified to yield new products. Given the synergy observed among two-component lantibiotics, which display similar or higher activity than the best single-component lantibiotic nisin (Morgan, et al. (2005) supra), the engineering of new lantibiotics with therapeutic potential could be realized.

### SUMMARY OF THE INVENTION

The present invention is a two-component Bacillus lantibiotic composed of the amino acid sequences set forth in SEQ ID NO:1 and SEQ ID NO:2. Pharmaceutical compositions containing said lantibiotic, as well as nucleic acid molecules, vectors, and host cells expressing said lantibiotic are also provided.

The present invention is also a method for producing the two-component Bacillus lantibiotic of the present invention. The method involves contacting precursor peptides containing amino acids sequences set forth in SEQ ID NO:1 and SEQ ID NO:2 with at least one modifying enzyme capable of effecting dehydration and cyclization of the precursor peptide, and cleaving the leader peptide from the precursor peptides thereby producing a biologically active two-component Bacillus lantibiotic.

The present invention further relates to a Bacillus lantibiotic modifying enzyme which effects dehydration and cyclization of a peptide or polypeptide and a method for using the same to modify a peptide or polypeptide. Nucleic acid molecules, vectors, and host cells expressing said lantibiotic modifying enzymes are also provided.

The present invention is also a kit for producing haloduracin, wherein said kit contains precursor peptides HalA1 and HalA2 and modifying enzymes HalM1 and HalM2.

Methods for preventing or inhibiting the growth of a bacterium and preventing or treating a bacterial infection using an effective amount of the two-component Bacillus lantibiotic of the present invention are also provided.

### BRIEF DESCRIPTION OF THE DRAWINGS

FIGS. 1A-1C show the sequence alignments of HalA1 and HalA2 (FIG. 1A); HalA1 with the α prepeptides from plantaracin W (PlwAα), staphylococcin C55 (SacAα) , lacticin 3147 (LtnA1), BhtA1, and SmbA1 (FIG. 1B); and of HalA2 with the β prepeptides from plantaracin W (PlwAβ), lacticin 3147 (LtnA2), BhtA2, SmbA2, SacAβ, and the two peptides of cytolysin (CylL-AS and CylL-AL)(FIG. 1C). Serine and threonine residues in the structural regions are underlined, as are the cysteine residues that may be involved in lanthionine thioether formation. The conserved protease cleavage sequences are boxed.

FIG. 2 shows the amino acid sequence of HalA1 (FIG. 2A) or HalA2 (FIG. 2B) from B. halodurans aligned with the amino acid sequence of the lantibiotic alpha (FIG. 2A) or beta (FIG. 2B) peptide from B. licheniformis.

FIG. 3 shows the proposed structures for the Hall (SEQ ID NO:5) and Hal2 (SEQ ID NO:6) peptides of the two-component lantibiotic haloduracin. The closest structural analogs, the alpha peptide from plantaricin, Plwα (SEQ ID NO:53), and the Ltn2 peptide (SEQ ID NO:54) from lacticin 3147, are shown for comparison. Dehydrobutyrine (Dhb) and Dehydroalanine (Dha) residues are indicated. MeLan and Lan bridges are indicated as are cystine linkages. Rings in Hal peptides are indicated by letters to correspond to discussion in the Examples.

### DETAILED DESCRIPTION OF THE INVENTION

Lantibiotics are ribosomally synthesized peptides that undergo post-translational modifications to their mature, antimicrobial form. They are characterized by the presence of the unique amino acid lanthionine, which is introduced via dehydration of Ser/Thr residues followed by reaction of the resulting dehydro amino acids with cysteines to form thioether linkages. Two-component lantibiotics utilize two peptides that are each post-translationally modified to yield two functionally distinct products that act in synergy to provide bactericidal activity. For the purposes of the present invention, the term peptide is intended to embrace a string of amino acid residues of 100 amino acids in length, wherein the term polypeptide or protein generally refers to molecules of greater than 100 amino acids in length.

Novel two-component lantibiotics from Bacillus sp. have now been identified. Haloduracin, encoded by the genome of the Gram-positive alkaliphilic bacterium Bacillus halodurans C-125, was heterologously expressed and the purified precursor peptides, HalA1 and HalA2, were processed by the expressed and purified modification enzymes HalM1 and HalM2 in an in vitro reconstitution assay. The activity of each HalM enzyme was substrate-specific and the assay products exhibited antimicrobial activity after removal of their leader sequences at an engineered Factor Xa cleavage site, indicating that correct thioether formation had occurred. Haloduracin's biological activity was dependent on the presence of both modified peptides and was comparable to the bactericidal effects exhibited by the peptides isolated from the producer strain. The structures of the two mature haloduracin peptides, Hal1 and Hal2, were determined and have similarities as well as some distinct differences compared to other known two-component lantibiotics.

Moreover, HalA1 and HalA2 exhibit sequence identity (39.2% and 35.6%, respectively) with lantibiotic alpha and beta peptides encoded by Bacillus licheniformis. Similar to the haloduracin gene cluster, B. licheniformis encodes two prepeptides, two modification enzymes, and several additional transport, immunity, and regulation proteins involved in lantibiotic biosynthesis. Of significance is the nearly identical C-termini of the mature B. halodurans and B. licheniformis lantibiotic peptides. Wherein the alpha peptides share the common amino acid sequence Cys-Thr-Xaa₁-Thr-Xaa₂-Glu-Cys-Met-Pro-Ser-Cys-Asn (SEQ ID NO:1), wherein Xaa₁ is an aliphatic amino acid residue (e.g., Ile, Val, or Leu) and Xaa₂ is any amino acid residue; the beta peptides share the common amino acid sequence Leu-Cys-Pro-Thr-Thr-Lys-Cys-Thr-Ser-Xaa₁-Cys (SEQ ID NO:2), wherein Xaa₁ is Gln or Arg.

Accordingly, the present invention is a two-component Bacillus lantibiotic composed of alpha and beta peptides comprising the amino acid sequences set forth in SEQ ID NO:1 and SEQ ID NO:2, respectively. As used herein, the term “lantibiotic” refers to a biologically active compound that acts so as to modify the ability of a target organism to develop, grow, proliferate, or otherwise function. The term can optionally include a compound derived by genetic engineering techniques, synthetic techniques, or a combination of techniques. For example, a lantibiotic can be at least partially synthetic and at least partially recombinant; thus the term can include variants of natural lantibiotics.

The term “target organism” refers to bacteria, viruses, fungi, or protozoa. Target organisms can also include a mammal, particularly a human. In the case of a multicellular organism such as a human, the term is meant to broadly convey a cell, tissue, organ, or fluid of the organism, whether in vivo, ex vivo, or in vitro. In a particular embodiment, the target organism is a bacterium and the compound acts to reduce or control growth or proliferation of the bacterium.

Two-component Bacillus lantibiotics include the haloduracin alpha and beta peptides isolated from B. halodurans, as well as the alpha and beta peptides of the lantibiotic isolated from B. licheniformis. In one embodiment, the present invention provides the B. halodurans alpha and beta haloduracin prepeptides (i.e., HalA1 and HalA2) set forth as SEQ ID NO:3 and SEQ ID NO:4, respectively. In another embodiment, the present invention provides the mature B. halodurans alpha and beta haloduracin peptides (i.e., Hal1 and Hal2) set forth as SEQ ID NOs:5 and 6, respectively. The haloduracin alpha peptide is composed of 28 amino acid residues in its mature form and has a molecular weight of 2332 Da, whereas the beta peptide is first processed to a 30 amino acid residue peptide which is subsequently further cleaved to 24 amino acid residues in its mature form having a molecular weight of 3046 Da. In yet another embodiment, the present invention provides the B. licheniformis alpha and beta lantibiotic prepeptides set forth as SEQ ID NOs:7 and 8, respectively. In still another embodiment, the present invention provides Bacillus alpha and beta peptides containing an exogenous protease cleavage sequence such as that recognized by Factor Xa, i.e., Ile-Glu-Gly-Arg (SEQ ID NO:9). Exemplary haloduracin alpha and beta peptide amino acid sequences containing an exogenous protease cleavage sequence are set forth herein as SEQ ID NO:10 and SEQ ID NO:11, respectively. Moreover, it is contemplated that the lantibiotic subunits are interchangeable, e.g., an alpha subunit of haloduracin can be combined with a beta subunit of the B. licheniformis lantibiotic to produce a biologically active lantibiotic.

The two-component Bacillus lantibiotics of the present invention can be isolated and purified from the respective Bacillus species which naturally produce the desired lantibiotic using methods as exemplified herein or well-known in the art of lantibiotic purification; expressed in a heterologous system (e.g., E. coli) via the nucleic acid molecules disclosed herein; produced via in vitro translation, or chemically synthesized using established methods. As used herein, the term “purified” refers to a molecule having been separated from a cellular component.

Whether produced in vitro, in vivo or chemically synthesized, the instant lantibiotic peptides can be composed of natural, non-proteinogenic, unnatural or derivatized amino acid residues. In the context of the present invention, a natural amino acid includes one of the 20 naturally occurring amino acid residues (i.e., alanine, arginine, asparagine, aspartic acid, cysteine, glutamic acid, glutamine, glycine, histidine, isoleucine, leucine, lysine, methionine, phenylalanine, praline, serine, threonine, tryptophan, tyrosine, valine), whereas the term “derivatized amino acid” refers to any amino acid that is derivatized chemically or biosynthetically. An example of a derivatized amino acid is selenocysteine. Further, the term “non-proteinogenic amino acid” as used in the context of the present invention refers to an amino acid that is not incorporated by normal in vivo biosynthesis into a protein and “unnatural amino acid” refers to a synthetic amino acid or refers to an amino acid that is typically foreign to a particular organism. Unnatural amino acids can optionally be a subset of non-proteinogenic amino acids.

By way of illustration, a synthetic biologically active lantibiotic containing at least one non-proteinogenic amino acid, unnatural amino acid, peptoid, beta amino acid, or derivatized amino acid can be produced by generating a first precursor lantibiotic peptide; generating a second precursor lantibiotic peptide, wherein said second precursor lantibiotic peptide contains at least one unnatural amino acid, peptoid, or derivatized amino acid; and combining said first and second precursor lantibiotic peptides so as to produce a third precursor lantibiotic peptide which contains the at least one unnatural amino acid, peptoid, or derivatized amino acid. In such an approach, the step of combining the precursor peptides can include ligation, conjugation, or other connection of said first precursor peptide to said second precursor peptide. A synthetic biologically active lantibiotic thus produced can be further reacted with an effective amount of a purified modifying enzyme as disclosed herein to effect dehydration and cyclization of the third precursor peptide. The leader peptide of the precursor lantibiotic peptide can than be cleaved using a suitable protease.

In this regard, it has been demonstrated that His6-LctA(1-38) and His6-LctA(1-38)Cys38Sec can be produced by expressed protein ligation (EPL) (Reis, et al. (1994). Appl. Environ. Microbiol. 60:2876-83) of the His6-LctA(1-37)-intein-CBD fusion with cysteine and selenocysteine (Sec), respectively. See U.S. patent application Ser. No. 11/034,275. Thus, it is contemplated that like LctM, semisynthetic Bacillus lantibiotic peptide substrates generated by EPL will be recognized by HalM1 and HalM2 for lantibiotic engineering of haloduracin as well as other lantibiotics including subtilin and nisin.

Given the substrate promiscuity of lantibiotic modifying enzymes such as LctM, it is contemplated that HalM1 and HalM2 can also be used in the production of novel lantibiotics. To demonstrate this, steric and electronic tolerance of the enzymes is assessed. This is followed by the incorporation of amino acids designed to answer specific questions about the post-translational modification process including mutants that incorporate peptide fragments from other lantibiotic prepeptides. The structural diversity accessible by these studies is greatly increased by using semi-synthetic substrates prepared by combinatorial parallel synthesis. In addition to the fundamental scientific knowledge that comes forth from these studies, they allow access to molecules with interesting properties that are not easily prepared by either chemical or biological techniques.

Thus, the present invention also relates to isolated and purified nucleotide sequences encoding the Bacillus lantibiotics disclosed herein. In one embodiment, the present invention provides the nucleic acid molecules set forth in SEQ ID NOs:12 and 13 which encode the B. halodurans alpha and beta haloduracin prepeptides (i.e., HalA1 and HalA2), respectively. In another embodiment, the present invention embraces nucleic acid molecules which encode alpha and beta subunits of the B. licheniformis two-component lantibiotic (i.e., SEQ ID NOs:14 and 15) In another embodiment, the present invention provides for nucleic acid molecules encoding lantibiotic modifying enzymes. Exemplary HalM1 and HalM2 nucleic acid molecules are set forth in SEQ ID NOs:16 and 17, whereas exemplary nucleic acid molecules encoding LanM1 and LanM2 are set forth in SEQ ID NOs:18 and 19).

Modifications to the nucleic acids of the present invention are also contemplated as long as the essential structure and function of the peptide or polypeptide encoded by the nucleic acids are maintained. Likewise, fragments used as primers or probes can have substitutions as long as enough complementary bases exist for selective, specific hybridization with high stringency.

Modifications of the peptides or polypeptides specifically disclosed herein, include amino acid substitutions based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are employed.

In making amino acid substitutions, the hydropathic index of amino acids can be considered. The importance of the hydropathic amino acid index in conferring interactive biologic function on a protein is generally understood in the art (see, Kyte and Doolittle (1982) J. Mol. Biol. 157:105). It is accepted that the relative hydropathic character of the amino acid contributes to the secondary structure of the resultant protein, which in turn defines the interaction of the protein with other molecules.

Polymorphisms are also embraced by the present invention. Polymorphisms are variants in the gene sequence. They can be sequence shifts found between various bacterial strains and isolates which, while having a different sequence, produce functionally equivalent gene products. Polymorphisms also encompass variations which can be classified as alleles and/or mutations which can produce gene products which may have an altered function. Polymorphisms also encompass variations which can be classified as alleles and/or mutations which either produce no gene product, an inactive gene product, or increased levels of gene product.

As exemplified herein, nucleic acid molecules of the present invention can be expressed separately, i.e., inserted into separate vectors for expression and purification of individual gene products, namely alpha and beta peptides and modifying enzymes, or alternatively collectively (e.g., as a gene cluster) inserted into a vector as an expression cassette. The nucleic acid molecules of the invention can encode for alpha and beta peptides and modifying enzymes as well as fusion proteins thereof. Fusion proteins include fusions with a heterologous polypeptide or peptide, i.e. a signal sequence for secretion and/or other polypeptide which will aid in the purification of peptide or polypeptide (e.g., GST, His6, or the like). Such vectors are known or can be constructed by those skilled in the art and generally contain all expression elements (e.g., promoters, terminator fragments, enhancer elements, marker genes and other elements as appropriate) necessary to achieve the desired transcription of the sequences. Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses such as bacteriophages, baculoviruses, and retroviruses, DNA viruses, cosmids, plasmids, and other recombination vectors. The vectors can also contain elements for use in either prokaryotic or eukaryotic host systems. One of ordinary skill in the art will know which host systems are compatible with a particular vector.

The vectors can be introduced into cells or tissues and expressed by any one of a variety of known methods within the art. Such methods can be found generally described in Sambrook et al. (1989, 1992) Molecular Cloning: A Laboratory Manual, Cold Springs Harbor Laboratory, New York; Ausubel et al. (1989) Current Protocols in Molecular Biology, John Wiley and Sons, Baltimore, Md.; Chang, et al. (1995) Somatic Gene Therapy, CRC Press, Ann Arbor, Mich.; Vega, et al. (1995) Gene Targeting, CRC Press, Ann Arbor, Mich.; Vectors: A Survey of Molecular Cloning Vectors and Their Uses, Butterworths, Boston, Mass. (1988); and include, for example, stable or transient transfection, lipofection, electroporation and infection with recombinant viral vectors. Introduction of nucleic acids by infection offers several advantages over other listed methods. Higher efficiencies can be obtained due to their infectious nature. Moreover, viruses are very specialized and typically infect and propagate in specific cell types. Thus, their natural specificity can be used to target the vectors to specific cell types in vivo or within a tissue or mixed culture of cells. The viral vectors can also be modified with specific receptors or ligands to alter target specificity through receptor mediated events.

Host cells suitable for introduction and expression of the nucleic acids of the invention are desirably bacterial; however, yeast (e.g., Pichia, Saccharomyces, etc.), mammalian, or insect host cells are also contemplated as is a cell-free expression system. In particular embodiments, the host cell or culture is bacterial. Exemplary bacterial host cells include E. coli as well as Bacillus sp.

As will be understood by the skilled artisan upon reading the instant disclosure, precursor lantibiotic peptides are generally first modified and then become biologically active (i.e., they prevent or inhibit the growth of a target organism) by proteolytic cleavage of the leader peptide from the mature peptide; wherein cleavage can occur prior to, concomitantly with or after export from the cell. Therefore, the alpha and beta lantibiotic precursor peptides can be expressed or synthesized with an endogenous protease cleavage sequence; or expressed or synthesized with an exogenous protease cleavage sequence which can be cleaved by a selected protease, e.g., Factor Xa, thereby effecting removal of the leader peptide. In this regard, the instant lantibiotic precursor peptides can proteolytically processed in vivo or processed in vitro under controlled conditions. Alternatively, the mature form of the peptides can be directly expressed or synthesized. As such, in one embodiment, an intact leader or structural peptide is not essential. For example, it is contemplated that the leader peptide can be combined with the mature form of the lantibiotic peptide in trans to facilitate the dehydration and cyclization of the mature peptide by a modifying enzyme.

Likewise, enzymes that modify the instant lantibiotic peptides (e.g., HalM1 and HalM2 or LanM1 and LanM2) can be co-expressed by a recombinant host cell which expresses the alpha and beta peptides to provide in vivo modification of the peptides, or alternatively the modifying enzymes can be provided in an in vitro reconstitution reaction to modify the alpha and beta peptides. Accordingly, contact of a lantibiotic peptide with a modifying enzyme specifically encompasses both in vivo and in vitro embodiments.

A Bacillus lantibiotic modifying enzyme of the present invention refers to a polypeptide or fragment thereof capable of acting upon an alpha or beta lantibiotic peptide so as to effect both at least one dehydration reaction and at least one cyclization reaction. In this regard, the present invention also provides a purified modifying enzyme capable of producing a biologically active lantibiotic peptide by effecting dehydration and cyclization of a precursor peptide. In one embodiment, the Bacillus lantibiotic modifying enzyme is a HalM1 enzyme. In another embodiment, the Bacillus lantibiotic modifying enzyme is a HalM2 enzyme. In particular embodiments, the Bacillus lantibiotic modifying enzyme is HalM1 or HalM2 having an amino acid sequence as respectively set forth in SEQ ID NOs:20 and 21. In other embodiments, Bacillus lantibiotic modifying enzymes are obtained from B. licheniformis (i.e., LanM1 and LanM2), the amino acid sequence of which are set forth herein as SEQ ID Nos:22 and 23, respectively. Still other embodiments contemplate the use of CinM (cinnamycin LanM), MrsM (mersacidin LanM), MutM (mutacin II LanM), ScnM (streptococcin A-FF22 LanM), RumM (ruminococcin A LanM), LtnM1 and LtnM2 (lacticin 3147 LanM), LctM (lacticin 481 LanM), or NukM modifying enzymes to effect dehydration and cyclization of the instant two-component Bacillus lantibiotics.

As has been demonstrated (Sahl and Bierbaum (1998) Annu. Rev. Microbiol. 52:41-79), the proteins which are involved in post-translational processing and modification of lantibiotics can be used in vitro to modify other polypeptides or peptides (especially other lantibiotics) and increase the stability of such molecules. As such, particular embodiments embrace the use of a Bacillus HalM1 or HalM2 enzyme or a LanM1 or LanM2 enzyme to modify HalA1 or HalA2 (i.e., haloduracin) peptides; or LanA1 or LanA2 peptides as well as other polypeptides and lantibiotics. By way of illustration, such a method involves contacting a primary translation product of another lantibiotics (e.g., duramycin) with a modifying enzyme of the invention so that the modifying enzyme effects dehydration and cyclization of the lantibiotic. Such a method can be carried our in vitro, using the translation products, or in vivo, e.g., by introducing the structural gene for another lantibiotic into a host cell which expresses HalM1 or HalM2 enzyme or LanM1 or LanM2 enzyme.

Having demonstrated in vitro reconstitution of HalM1 and HalM2 for producing haloduracin, the present invention also relates to a kit containing precursor peptides HalA1 and HalA2 in combination with modifying enzymes HalM1 and HalM2 for producing haloduracin. Alternatively, the kit can contain B. licheniformis alpha and beta peptides in combination with LanM1 and LanM2 modifying enzymes. The kit can further contain buffers suitable for carrying out dehydration and cyclization of the precursor peptides and an instruction manual. In some embodiments, the alpha and beta precursor peptides contain exogenous protease cleavage sequences and the kit further contains a selected protease which recognizes and cleaves the exogenous protease cleavage sequence.

Using the in vitro biosynthesis system disclosed herein, antimicrobial peptide design and engineering is now possible. The in vitro biosynthesis system allows detailed investigation of the substrate specificity of each individual modifying enzyme as site-directed mutants are readily and rapidly accessible through combinatorial methods. Evaluation of substrate specificity in vitro has advantages over in vivo methods for a complex system like lantibiotic biosynthesis. In particular, when a lantibiotic producing strain shows low or no bioactivity after mutation of the substrate peptide, this can imply the original residue was critical for biological activity, or alternatively it may be due to failure of the biosynthetic proteins (dehydratase, cyclase, or protease), poor expression of the mutant substrates, inability of the wild-type transport proteins to secrete the processed mutants, breakdown of the analog products, or potential toxicity of the non-natural products to the producing strain due to non-recognition of the mutants by the innate immunity proteins. As such, substrate specificity of the biosynthetic proteins can be examined in a much more controlled fashion, and improved lantibiotic variants thus identified can be invaluable starting points to subsequently engineer and optimize an in vivo production system.

In particular, it is contemplated that non-silent mutations of the alpha or beta lantibiotic peptides will produce changes in the amino acid sequence leading to a variant form of two-component lantibiotic having one or more different properties compared to naturally occurring lantibiotic. Similarly, mutations of the modifying enzymes can lead to different post-translational modifications. Such mutagenesis can be performed using available methods, e.g., chemical mutagenesis, alanine-scanning mutagenesis, site-directed mutagenesis using oligonucleotides, error-prone PCR or by propagating target nucleic acid in an appropriate plasmid in a mutator strain, e.g., the XL1-Red strain of E. coli (STRATAGENE). The protocol for this procedure is described in Greener and Callahan (1993) STRATEGIES 6:32-34. Mutagenesis can be carried out on a particular coding sequence (e.g., HalA1, HalA2, HalM1, or HalM2) or the entire gene cluster encoding the biosynthetic machinery for lantibiotic production can be subjected to mutagenesis.

In some embodiments, the present invention provides a method for producing a library of lantibiotic-producing host cells, wherein the host cells produce mutant forms of haloduracin. Such a library can be screened for desirable properties. Desirably, the library is initially screened for lantibiotic production (e.g. by determining the effect on Lactococcus lactis growth), and then screened for interesting and/or advantageous mutations. Subsequent screening can be limited, e.g., following such an initial screening step, to host cells which display lantibiotic production. Lantibiotics identified by such a screening method can be purified and used in accordance with the pharmaceutical compositions and therapeutic methods disclosed herein.

Having demonstrated the isolation and production of a biologically active two-component lantibiotic from Bacillus, the present invention also relates to two-component Bacillus lantibiotic compositions and methods for use in pharmaceutical, agricultural, and food industry applications to combat infections caused by strains of Actinobacilli, Clostridium sp., Corynebacteria, Enterococci, Listeria monocytogenes, Mycobacterium phlei, Neisseria, Propionibacterium, Staphylococci, Streptococci, and other Gram-positive bacteria. In this regard, the present invention relates to methods for preventing or inhibiting the growth of a bacterium and preventing or treating a bacterial infecting by providing an effective amount of a two-component Bacillus lantibiotic disclosed herein. Such an effective amount provides a measurable reduction or inhibition in the growth or proliferation of the bacterium.

Thus according to particular embodiments of the present invention, the instant two-component Bacillus lantibiotic is provided in pharmaceutical compositions containing, as active ingredient, the lantibiotic in admixture with one or more pharmaceutical carriers and/or excipients. The term pharmaceutical composition as used herein is meant to cover human treatment and prophylaxis as well as the veterinary field. Treatment of animals such as cow (mastitis), chicken and the like are within the scope of the present invention.

For pharmaceutical administration, the two-component Bacillus lantibiotic can be incorporated into preparations in either liquid or solid forms using carriers and excipients conventionally employed in the pharmaceutical art, optionally in combination with further active ingredients. The preparation can, for example, be applied orally, parenterally enterally or preferably topically. Preferred forms include, for example, solutions, emulsions, gels, sprays, lotions, ointments, creams or powders. A generally recognized compendium of such preparations is Remington: The Science and Practice of Pharmacy, Alfonso R. Gennaro, editor, 20th ed. Lippincott Williams & Wilkins: Philadelphia, Pa., 2000. The carrier(s) or excipient(s) selected must be acceptable in the sense of being compatible with the other ingredients of the formulation and not injurious to the subjected receiving treatment.

It is contemplated that one of ordinary skill can readily determine the amount of two-component Bacillus lantibiotic to be administered. It is apparent that the dosage will be dependent on the particular treatment used. It should also be clear that the dosage should be chosen to display the biological activity without causing adverse effects. It will be understood that age, sex, type of disease, of formulation and other variables known to the person of ordinary skill will affect determination of the dosage to be used.

Advantageously the pharmaceutical compositions can be formulated as dosage units, each unit being adapted to supply a fixed dose of active ingredient. The total daily dose can, of course, be varied depending on the subject treated and the particular use of the composition. Such adjustment can be readily made by the skilled clinician or veterinarian.

If under certain conditions, it would be beneficial to provide a subject with a longer circulating time and/or slow release of the two-component Bacillus lantibiotic, the lantibiotic can be trapped in well-known delivery molecules such as liposomes, synthetic vesicles, nanoerythrosomes (U.S. Pat. No. 5,653,999) and the like, according to known methods.

In foodstuff compositions, wherein the instant two-component Bacillus lantibiotic prevents solid or liquid food from spoiling (i.e. meats, dowry products, beer, wine and the like) by inhibiting or killing bacteria and especially harmful bacteria, it is contemplated that the instant lantibiotic can be added directly to the food. Furthermore, the instant lantibiotic can be used as biopreservative agent in foods and in personal hygiene products as well as a anticarries agent (i.e., in toothpaste, mouth wash, and in topical application), disinfectant cleanser (to combat acne for example), selective agent against Gram-positive bacteria in culture media (Ray (1992) In: Food Biopreservative of Microbial Origin, Ray et al. (Eds) CRC Press Inc., Boca Raton, Fla., p. 207-264; Harlanda (1993) In: Bacteriocins of Lactic Acid Bacteria, Hoover et al (Eds.) Acad. Press Inc., San Diego, Calif., p. 63-91; U.S. Pat. No. 5,231,013).

In some embodiments, the instant lantibiotic is provided as a prodrug. The term “prodrug” refers to compounds that are rapidly transformed in vivo to yield the parent compound, for example, by hydrolysis in blood. A thorough discussion is provided in Higuchi and Stella, Pro-drugs as Novel Delivery Systems, Vol. 14 of the A.C.S. Symposium Series, and in Bioreversible Carriers in Drug Design (1987) Edward B. Roche (ed.) American Pharmaceutical Association and Pergamon Press.

Such a prodrug includes esters or amides of the instant alpha and beta peptides. Examples of pharmaceutically acceptable, non-toxic esters of said peptides include C₁-C₆ alkyl esters wherein the alkyl group is a straight or branched chain. Acceptable esters also include C₅-C₇ cycloalkyl esters as well as arylalkyl esters such as, but not limited to benzyl. As will be appreciated by the skilled artisan, such esters can be prepared according to conventional methods.

Examples of pharmaceutically acceptable, non-toxic amides of the lantibiotic peptides of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. As with the esters, amides of the instant alpha and beta peptides can be prepared according to conventional methods.

The invention is described in greater detail by the following non-limiting examples.

### EXAMPLE 1

### Materials

Bacillus halodurans C-125 was purchased from the American Type Culture Collection (ATCC, Manassas, Va.). The bioactivity indicator strain Lactococcus lactis CNRZ 117 was obtained from the Centre National de Recherches Zootechniques (Jouy-enJosas, France). Genomic DNA isolated from B. halodurans C-125 was also purchased from the ATCC. Chemically competent Escherichia coli DH5α cells were purchased from the UIUC Cell Media Facility, while electrocompetent BL21 (DE3) cells were purchased from STRATAGENE (La Jolla, Calif.). Media was obtained from DIFCO Laboratories (Sparks, Md.). Oligonucleotide primers were synthesized by Operon Technologies (Alameda, Calif.). Cloned Pfx polymerase, T4 DNA ligase, and restriction enzymes NdeI, NheI, BamHI, and XhoI were obtained from INVITROGEN (Carlsbad, Calif.). Factor Xa was obtained from NEW ENGLAND BIOLABS (Ipswich, Mass.). Cloning vectors (pET) were purchased from NOVAGEN (Madison, Wis.). Iodoacetamide was obtained from Acros Organics (Geel, Belgium). DTT (1,4 dithio-DL-threitol) was purchased from FISHER BIOTECH (Hampton, N.H.) and TCEP (tris(2-carboxyethyl) phosphine hydrochloride) was obtained from Sigma-Aldrich (St. Louis, Mo.). IPTG (isopropyl-1-thio-Dgalactopyranoside) was obtained from CALBIOCHEM (San Diego, Calif.). C-18 zip tips were purchased from MILLIPORE (Billerica, Mass.). Gel extraction, plasmid mini-prep, and PCR purification kits were purchased from QIAGEN (Valencia, Calif.). A 5 mL HITRAP chelating HP column and PD-10 columns were purchased from GE Healthcare. Thiopropyl SEPHAROSE resin was purchased from Amersham Biosciences (Piscataway, N.J.). Dialysis tubing (SPECTRA/POR) was obtained from Spectrum Laboratories, Inc. (Rancho Dominguez, Calif.). The ketone modifying agents 1,2-phenylenediamine and benzoyl hydrazine were purchased from Sigma-Aldrich and Alfa Products, Thiokol/Ventron Division (Danvers, Mass.), respectively.

### EXAMPLE 2

### Methods

Induction of Haloduracin Production. B. halodurans C-125 was obtained as a freeze-dried culture and rehydrated using trypticase soy broth (pH 7 and 9) under aerobic conditions at 37° C. A 5 mL culture of B. halodurans C-125 was inoculated from this cell stock in LB broth and grown under aerobic conditions for 30 hours at 37° C. Aliquots of the culture (100 μL) were removed and plated on modified nutrient agar. The plates were grown an additional 90 hours at 30° C. until a dense lawn of bacteria was present. Bacterial lawns were gently washed with sterile water to remove the cells from the plate. The cell suspension was collected and incubated overnight at 30° C. without shaking to further induce sporulation. The solution was then centrifuged at 5000×g for 30 minutes at 4° C. and the supernatant was filtered using a 0.2 μm syringe filter to remove any remaining cells or spores. The cell-free solution containing haloduracin was analyzed by mass spectrometry and used for bioassays.

Mass Spectrometry. Matrix-assisted laser desorption ionization-time of flight (MALDI-TOF) mass spectrometry was performed using a Voyager-DE-STR (APPLIED BIOSYSTEMS, Foster City, Calif.) instrument. Assay samples were prepared for MS by purification over a C-18 zip tip. The sample was eluted from the zip tip into a saturated solution of α-hydroxycinnamic acid prepared in 50% acetonitrile containing 0.1% trifluoroacetic acid (TFA) for analysis. High resolution ESI-FTMS was performed on a custom-built 8.5 T Quadrupole-FTMS (Miller, et al. (2006) J. Am. Chem. Soc. 128:1420-1; Patrie, et al. (2004) J. Am. Soc. Mass Spectrom. 15:1099-108). The fragment ion prediction program of the ProSight PTM software bundle was used to generate fragment ion masses.

Bioactivity Assay. The inhibitory activity of haloduracin isolated from the producing strain and prepared in vitro was assayed using the solid agar medium test. Liquid molten GM17 agar (4% M17, 0.5% glucose, 1.5% agar) was cooled to 50° C. and seeded with an overnight culture of the indicator strain Lactococcus lactis CNRZ 117. After agar solidification in a Petri dish, samples were applied to a small well created in the medium. Assay samples were typically concentrated to dryness using a speed vac and rehydrated in a small volume (5-15 μL) of sterile water for application purposes. The cell-free solution isolated from B. halodurans C-125 was applied directly to the plate without further concentration. Plates were incubated overnight at room temperature and zones of inhibition were observed the next day.

Molecular Cloning of Haloduracin Expression Constructs. Genomic DNA from B. halodurans C-125 was used as the template for PCR amplification of halA1, halA2, halM1, and halM2. Primers (Table 1) were constructed that added an NdeI restriction site 5′ and XhoI restriction site 3′ to each halA gene. An NheI restriction site was added at the 5′ end of halM1, while an XhoI site was added to the 3′ end. The halM2 gene was amplified with an XhoI restriction site at the 5′ end and a BamHI restriction site at the 3′ end. The PCR products were digested with the appropriate restriction enzymes and gel purified using a QIAGEN gel extraction kit. Vector DNA (pET15b for halA1, halA2, halM2 and pET28b for halM1) digested with the same restriction enzymes was added to a ligation reaction containing T4 DNA ligase and the insert DNA. Chemically competent E. coli DH5α cells were transformed with each ligation mixture and plated on LB-agar containing the appropriate antibiotics to screen for positive clones (pET15b based constructs-ampicillin, 100 μg/mL; pET28b based constructs-kanamycin, 50 μg/mL). Clones were screened by redigestion of isolated plasmid DNA or colony PCR. Positive clones were confirmed by DNA sequence analysis.

TABLE 1

| | | | SEQ ID |
|---|---|---|---|
| Construct | Location | Primer Sequence | NO: |
| pHa1A1 | 5′ | GCGCCGCATATGACAAATCTT | 24 |
| | 3′ | AGGCTCGAGTTAGTTGCAAGA | 25 |
| pHa1A2 | 5′ | GCGCCGCATATGGTAAATTCA | 26 |
| | 3′ | AAACTCGAGTTAGCACTGGCT | 27 |
| pHa1M1 | 5′ | GCCGCTAGCATGAGAGAATTA | 28 |
| | 3′ | CGTCTCGAGTTAATGATTCGC | 29 |
| pHa1M2 | 5′ | GGGTATCCGCTCGAGATGAAAACTCCTCTAACAAGT | 30 |
| | 3′ | TATAAACGCGGATCCTTATCTGTCATGAATTCTCAA | 31 |
| pHa1A1-Xa | 5′ | ATTCTAGCTGGGATTGAAGGTCGTTGCGCATGGTAC | 32 |
| | 3′ | AGGCTCGAGTTAGTTGCAAGA | 33 |
| pHa1A2-Xa | 5′ | GCTTCAGGAGATATTGAAGGTCGTACAACTTGGCCT | 34 |
| | 3′ | AAACTCGAGTTAGCACTGGCT | 35 |

Engineering of a Factor Xa Cleavage Site in the HalA Peptides. To generate HalA peptides that contained a Factor Xa cleavage site, primers were designed for each peptide that contained the nucleotide sequence necessary to encode the amino acids Ile-Glu-Gly-Arg (SEQ ID NO:9) in place of four wild-type peptide residues. In the first round of PCR to generate a megaprimer for subsequent rounds, the mutations were encoded by the 5′ primer, while the 3′ primer remained the same as listed in Table 1. The template DNA used for this reaction was the pET15b construct containing the wild-type halA gene cloned previously. The double stranded PCR product of the first round was gel-purified and used as one of the primers in the next round of PCR. The other primer for round two was specific for the T7 promoter of the pET vector in which the gene was originally cloned. Using the megaprimer isolated above and the T7 promoter primer with the DNA of the pET construct containing the wild-type gene as template, a PCR product was generated that contained the appropriate mutations. In the case of HalA1, residues Val-Asn-Gly-Ala (SEQ ID NO:36) were replaced with Ile-Glu-Gly-Arg (SEQ ID NO:9) resulting in the sequence set forth in SEQ ID NO:10, while residues Val-His-Ala-Gln (SEQ ID NO:37) were substituted with Ile-Glu-Gly-Arg (SEQ ID NO:9) in HalA2 resulting in the sequence set forth in SEQ ID NO:11. The DNA was gel-purified and the modified gene of interest was excised from the PCR product by digestion with NdeI and XhoI. Following gel purification, each modified halA gene was ligated into pET15b digested with the appropriate restriction enzymes and transformed into E. coli DH5α cells. Positive clones were isolated and confirmed by DNA sequence analysis.

Overexpression and Purification of HalA Peptides. The electrocompetent E. coli BL21 (DE3) strain was transformed with the pET construct containing the appropriate N-terminal hexa-histidine halA fusion gene. Cultures were inoculated from single colony transformants and grown overnight at 37° C. in LB broth supplemented with 100 μg/mL ampicillin. The overnight culture was used to inoculate 3 liters of LB broth, and cells were grown at 37° C. to A600˜0.6-0.8. Expression was induced by the addition of 1 mM IPTG, and the culture was incubated at 37° C. for three additional hours. Cells were harvested by centrifugation at 6500×g for 20 minutes at 4° C. The pellet (˜15 grams) was resuspended in 30 mL of start buffer containing 20 mM sodium phosphate, pH 7.5, 20% glycerol, 500 mM NaCl, and 0.5 mM imidazole. The cell paste was subjected to sonication to lyse the cells. Cell debris was removed by centrifugation at 16,500×g for 20 minutes at 4° C. The supernatant was decanted and the pellet containing the insoluble peptide was resuspended in the same volume of start buffer. The sonication and centrifugation steps were repeated and the pellet was resuspended in 30 mL of buffer 1, containing 6 M guanidine hydrochloride, 20 mM sodium phosphate, pH 7.5, 500 mM NaCl, and 0.5 mM imidazole. The sample was sonicated and remaining insoluble material was removed by centrifugation at 16,500×g for 20 minutes at 4° C. and the supernatant passed through a 0.45 μm filter. The peptides were purified by immobilized metal affinity chromatography (IMAC) using a 5 mL Ni²⁺ column. The filtered sample was applied to the column and washed with two column volumes of buffer 1, followed by two column volumes of buffer 2 containing 4 M guanidine hydrochloride, 20 mM sodium phosphate, pH 7.5, 300 mM NaCl, and 30 mM imidazole. The peptide of interest was eluted in 1-2 column volumes of elution buffer containing 4 M guanidine hydrochloride, 20 mM sodium phosphate, pH 7.5, 100 mM NaCl, and 1 M imidazole. The fractions containing peptide were pooled and desalted via dialysis or reverse-phase high-performance liquid chromatography. Dialysis was performed using 1000 Da molecular weight cut off tubing in which the peptide sample buffer was exchanged with 20 mM sodium acetate, pH 4, followed by exchange with 0.05% HCl. Reverse-phase HPLC was performed on a C4 column using a gradient of 2-100% of 80% acetonitrile in 0.1% TFA. Following desalting by either method, the peptide sample was lyophilized to dryness and stored at −20° C.

Overexpression and Purification of the HalM Enzymes. Electrocompetent E. coli BL21 (DE3) strain was transformed with the pET construct containing the appropriate N-terminal hexa-histidine halM fusion gene. Cultures were inoculated from single colony transformants and grown overnight at 37° C. in LB broth supplemented with 100 μg/mL ampicillin or 50 μg/mL kanamycin. The overnight culture was used to inoculate 3 liters of LB broth, and cells were grown at 37° C. to A600 ˜0.5-0.6. Expression was induced by the addition of 1 mM IPTG, and the culture was incubated at 18° C. for ˜20 additional hours. Cells were harvested by centrifugation at 6500×g for 20 minutes at 4° C. The pellet was resuspended in 20 mM Tris, pH 7.6, 500 mM NaCl, and 10% glycerol and lysed by sonication at 65% intensity for 15 minutes. The sample was clarified by centrifugation at 16,500×g for 20 minutes at 4° C. to yield the crude cell-free extract, which was filtered through a 0.45 μm filter.

Each HalM protein was purified by IMAC using a 5 mL Ni²⁺ column. After the sample was applied to the column, it was washed with two column volumes each of 25 mM, 50 mM, and 75 mM imidazole in 20 mM Tris, pH 7.6, 500 mM NaCl, and 10% glycerol. The protein was eluted with two column volumes each of 200 mM and 500 mM imidazole in 20 mM Tris, pH 7.6, 500 mM NaCl, and 10% glycerol. Fractions were analyzed by SDS-PAGE and those containing protein were pooled and desalted using a PD-10 size exclusion column. The protein was stored in 20 mM Tris, pH 7.6, 100 mM or 500 mM KCl, and 10% glycerol at −80° C.

HalM Assays of HalA Substrates. Purified HalA peptides were incubated with purified HalM proteins in various combinations of substrates and enzymes in the presence of 50 mM MOPS, pH 7.2-7.5, 2.5 mM ATP, 1-3 mM TCEP, and 10 mM MgCl₂ at 25° C. for 2-4 hours. The final concentration of each peptide or protein was ˜0.4 mg/mL. Aliquots were removed at set times and subjected to purification over a C-18 zip tip followed by MALDI-TOF MS analysis.

Iodoacetoamide Modification of Haloduracin. The haloduracin peptides isolated from B. halodurans C-125 or produced in vitro were subjected to modification by iodoacetoamide (IAA). Hal1 and Hal2 isolated from B. halodurans were incubated with 5 or 10 mM IAA for 30-45 minutes at room temperature in the dark both before and after treatment with 1 mM TCEP to reduce any potential disulfide linkages. HalA1 and HalA2 were analyzed immediately following modification by the HalM enzymes, since excess reductant (TCEP) is present in the assay mixture and keeps all unreacted Cys reduced. Samples were taken for MALDI-TOF MS and excess IAA was removed by addition of ˜0.5 mg of thiolpropyl-SEPHAROSE resin in a water-slurry mixture and subsequent centrifugation.

Analysis for an N-terminal 2-Oxobutyryl Group by Diamine Modification. 1,2-Phenylenediamine was added to a 4 M sodium acetate buffer, pH 4.8 to a final concentration of 40 mM. Each peptide was added to this solution at a final concentration of 0.1-0.3 mg/mL for the Hal peptides or 0.03-0.1 mg/mL for the 2-oxobutyryl-Ala-Trp-Pro-Ser (SEQ ID NO:40) synthetic control peptide. Reactions were incubated at 38° C. for 12 hours and analyzed by MALDI-TOF MS. No change in mass was observed for either haloduracin peptide, while the AWPS peptide exhibited a decrease in mass of 84 Da (i.e., 566 Da to 482 Da).

Analysis for an N-terminal 2-Oxobutyryl Group by Hydrazine Modification. The Hal peptides isolated from B. halodurans C-125 or the positive control peptide Ala-Trp-Pro-Ser (SEQ ID NO:40) containing a 2-oxobutyryl moiety were incubated in 100 mM MOPS, pH 3 or 5 with 5 mM benzoyl hydrazine at a final concentration of 0.3 mg/mL for 12 hours at 25° C. Samples were analyzed by MALDTOF MS. No change in mass was observed for Hal1 or Hal2, indicating that hydrazone formation did not occur. In contrast, the Ala-Trp-Pro-Ser (SEQ ID NO:40) peptide exhibited a 118 Da increase in mass, consistent with hydrazone formation at the ketone functional group.

Factor Xa Cleavage of Peptide Leader Sequences. Factor Xa was used to remove the leader sequences from the HalA-Xa peptides following modification by the HalM enzymes. Both CaCl₂ and Factor Xa were added directly to the HalM assay mixture at final concentrations of 2 mM and 0.03 mg/mL, respectively. Samples were then incubated at room temperature for 3-6 hours to fully proteolyze the peptide substrates. Aliquots were removed for MALDI-TOF MS analysis. Reactions were concentrated to dryness using a speed vac and brought up in 10-15 μL of sterile water for use in the bioactivity assay.

### EXAMPLE 3

### Identification of Haloduracin

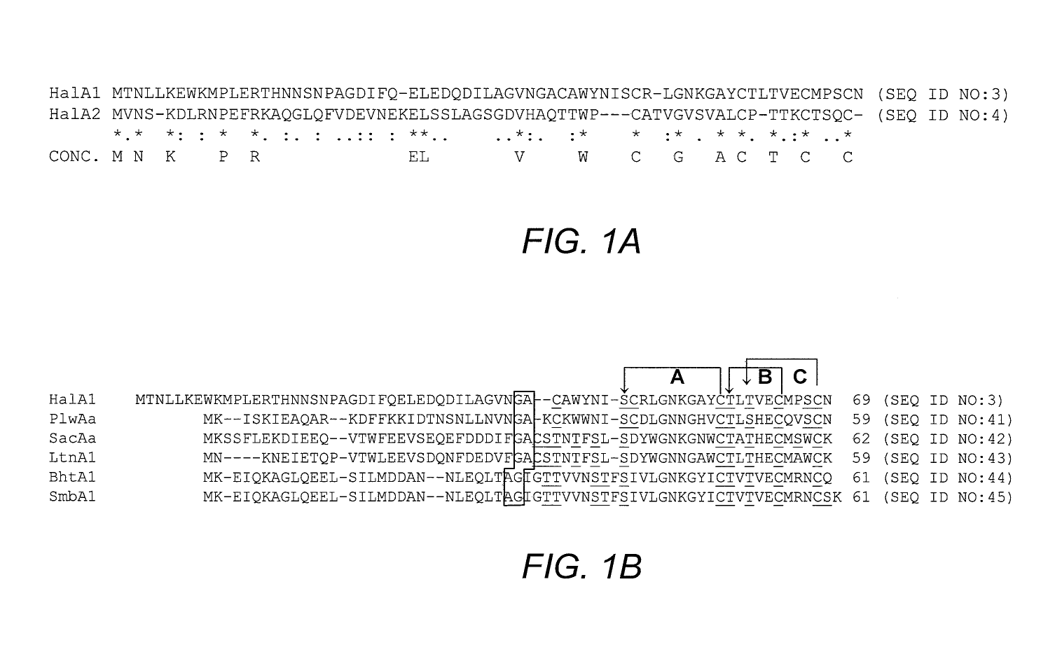

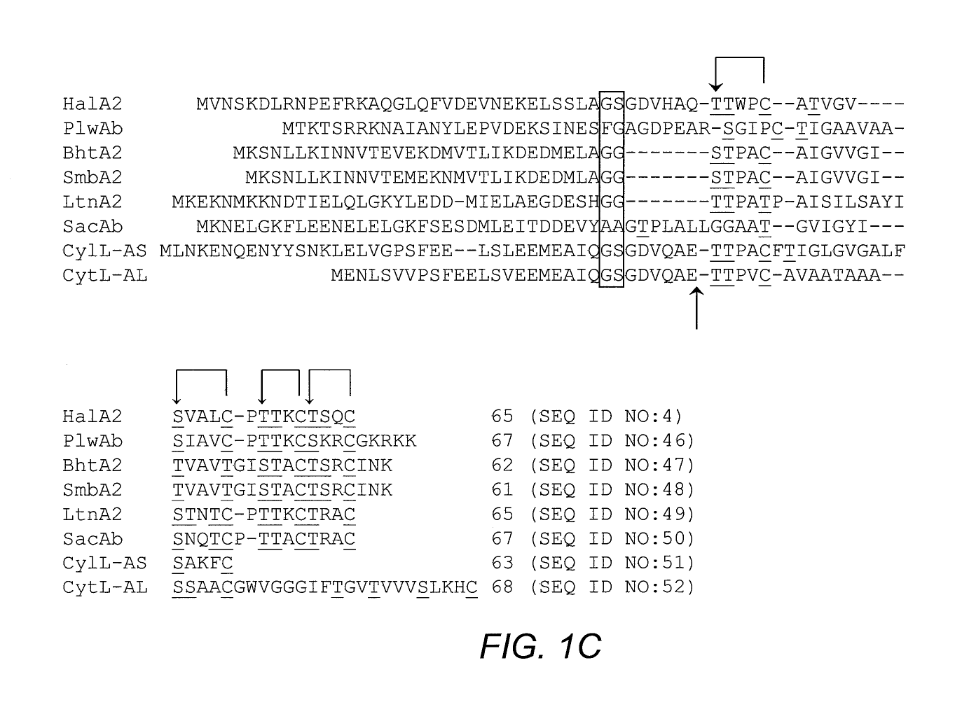

During a search for analogs of the lantibiotic mersacidin, a homolog of the mrsA gene was identified in the fully sequenced genome of the Gram-positive bacterium Bacillus halodurans C-125 (Takami, et al. (2000) Nucleic Acids Res. 28:4317-31). This strain had not previously been reported to produce a lantibiotic. The halA1 gene encoded a peptide of 69 residues (SEQ ID NO:3), with a 41-residue leader sequence (SEQ ID NO:38) of the double-glycine type that was expected to be removed by a protease resulting in a 28-residue active peptide (SEQ ID NO:5). The HalA1 peptide, found in GENBANK Accession No. BAB04173, shared 34% sequence identity with the precursor peptide for mersacidin. Further analysis of the surrounding DNA sequence identified the halA2 gene immediately 5′ of halA1. HalA2, found in GENBANK Accession No. BAB04172, contained 65 residues (SEQ ID NO:4), 35 of which likely encompassed the leader sequence (SEQ ID NO:39) based on a predicted double-glycine cleavage signal at residues Gly34-Ser35 resulting in a 24 amino acid residue active peptide (SEQ ID NO:6). The two prepeptides HalA1 and HalA2 shared 22.9% sequence identity with each other (FIG. 1A). HalA1 has significant sequence identity (40-50%) with peptides from other two-component systems, including LtnA1 (lacticin 3147; Ryan, et al. (1999) J. Biol. Chem. 274:37544-50), PlwAα (plantaricin W; Holo, et al. (2001) Microbiology 147:643-651), and SacAu (staphylococcin C55; Navaratna, et al. (1998) Appl. Environ. Microbiol. 64:4803-8) (FIG. 1B). HalA2 exhibits similarity (35-40% identity) to PlwAα (plantaricin W), CylL-L and CylL-S (cytolysin), and Ltn2 (FIG. 1C).

Inspection of the sequence alignments in FIG. 1B and FIG. 1C as well as the structures of lacticin 3147 and haloduracin reveals similarities and differences in the two component lantibiotics, which with the exception of lacticin 3147 (Weidemann, et al. (2006) Mol. Micro. 61:285-296), have not been structurally characterized. The A1/α-peptides all have the same topology of the three C-terminal rings, which is important in lipid II binding in mersacidin. On the other hand, the N-terminus is quite different amongst these peptides, with plantaricin and haloduracin both containing an N-terminal cyclic disulfide, lacticin 3147 and staphylococcin C55 an N-terminal methyllanthionine ring, and the very close homologs BHT and Smb lacking a ring altogether. The A2/β-peptides have structural motifs at both the N- and C-termini. The N-terminal methyllanthionine ring identified herein in Hal2 appears relatively common as sequence homology indicates it is present in all family members accept lacticin 3147 and staphylococcin C55 (FIG. 1C). This first ring is followed by a stretch of hydrophobic amino acids. It is in this region that the Ser to D-Ala conversions occur in LtnA2 of lacticin 3147. The next ring system (B-ring, FIG. 1C) is once again relatively conserved amongst currently known two-component lantibiotics, and is only absent in BHT/Smb. Finally, the two most C-terminal Lan/MeLan rings are conserved in all members that have a companion Al/α peptide. These rings are absent in the two peptides of cytolysin, with CylLs truncated after the B-ring and CylL_{L} containing an appended sequence that is unrelated to the other family members.

Two lanM genes, designated halM1 and halM2, were found flanking the two halA genes and appeared to encode the enzymes that perform the post-translational modification of HalA1 and HalA2. HalM1 (GENBANK Accession No. BAB04174) and HalM2 (GENBANK Accession No. BAB04171) exhibited 25% sequence identity to each other and other LanM proteins from both two-component and single component lantibiotic systems. The leader sequence of the modified HalA peptides was most likely removed by a bifunctional transport protein designated HalT that was also encoded in the gene cluster. HalT contains an N-terminal proteolytic region, six transmembrane regions, and an ATP binding domain and shares homology with the ATP-binding cassette (ABC) family of proteins (Havarstein, et al. (1995) Mol. Microbiol. 16:229-40). The fully modified biosynthetic products, designated Hal1 (from HalA1) and Hal2 (from HalA2), compose the lantibiotic haloduracin and were expected to act synergistically for bactericidal activity.

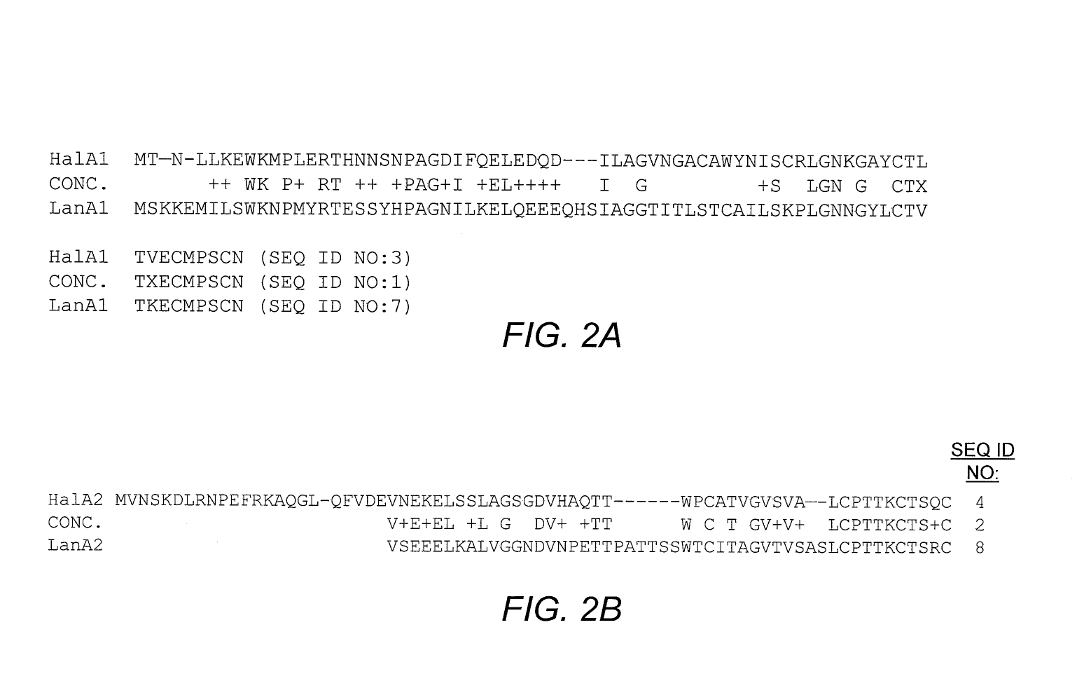

Including haloduracin, seven two-component lantibiotics have now been documented (Ryan, et al. (1999) J. Biol. Chem. 274:37544-50; Holo, et al. (2001) Microbiology 147:643-651; Navaratna, et al. (1998) Appl. Environ. Microbiol. 64:4803-8; Yonezawa & Kuramitsu (2005) Antimicrob. Agents Chemother. 49:541-8; Hyink, et al. (2005) FEMS Microbiol. Lett. 252:235-41). By searching the non-redundant database for homologs to the haloduracin peptides, another gene cluster in Bacillus licheniformis was identified that encoded two prepeptides designated herein as LanA1 and LanA2 (FIGS. 2A and 2B, respectively), two modification enzymes designated herein as LanM1 and LanM2, and several additional transport, immunity, and regulation proteins involved in lantibiotic biosynthesis. The amino acid sequence of the lantibiotic prepeptides of B. licheniformis as compared to that of haloduracin are depicted in FIGS. 2A and 2B. Bacillus licheniformis alpha prepeptide is found as GenBank Accession No. AE017333. The nucleotide sequences encoding LanA1, LanA2, LanM1, and LanM2 are set forth herein as SEQ ID NO:14, SEQ ID NO:15, SEQ ID NO:18, and SEQ ID NO:19, respectively.

### EXAMPLE 4

### Production of Haloduracin by B. halodurans C-125

Haloduracin production was observed when B. halodurans C-125 was grown on modified nutrient broth plates for >90 hours to induce sporulation, which often induces antibiotic production. The plates were washed with sterile water and incubated overnight. Cells and spores were then removed by centrifugation and the supernatant containing the haloduracin peptides was collected. Analysis of the cell-free supernatant by MALDI-TOF mass spectrometry (MS) indicated that two products with masses of 2332 Da (M+H) and 3046 Da (M+H) were present. When applied to a Lactococcus lactis CNRZ 117 indicator strain, a zone of growth inhibition was produced. This result demonstrated the isolation of active haloduracin from the producer strain under these conditions.

### EXAMPLE 5

### Expression and Purification of the Biosynthetic Components for Haloduracin Production

Genomic DNA isolated from B. halodurans C-125 was used as the template DNA for PCR amplification of halA1, halA2, halM1, and halM2. Each gene was cloned into the appropriate pET (NOVAGEN) vector to generate an N-terminal hexa-histidine (H6) fusion construct. The halA genes were overexpressed in E. coli and the corresponding peptides purified to homogeneity according to methods used for other lantibiotic prepeptides (Xie, et al. (2004) Science 303:679-81). Briefly, the peptides were expressed in inclusion bodies that were isolated by centrifugation and resolubilized in guanidinium hydrochloride. Each peptide was subsequently purified by immobilized metal affinity chromatography (IMAC) followed by reverse-phase high-performance liquid chromatography. Similarly, the halM genes were overexpressed in E. coli and the corresponding proteins purified to homogeneity by IMAC, resulting in proteins that were >90% pure.

Purified HalA1 and HalA2 were incubated together with purified HalM1 and HalM2 in an assay mixture containing TCEP (tris(2-carboxyethyl)phosphine hydrochloride), MgCl₂, and ATP (Xie, et al. (2004) supra; Chatterjee, et al. (2005) J. Am. Chem. Soc. 127:15332-3) and then subjected to MALDI-MS. Incubation of the prepeptides with both modification enzymes resulted in the 3-fold dehydration of HalA1 and the 7-fold dehydration of HalA2 by comparison to the peptide starting material. Based on the number of Ser/Thr residues in the proposed structural regions of HalA1 and HalA2 (FIG. 1), HalA1 underwent three of four possible dehydrations whereas HalA2 was dehydrated at seven of eight possible residues. These results were consistent with the haloduracin peptides isolated from the producer strain whose masses corresponded to the same number of dehydration events for each peptide.

The specificity of each enzyme was subsequently examined. Incubation of HalA1 with HalM1 generated a species that was dehydrated 3-fold, while incubation of HalA2 with HalM2 generated a species that was dehydrated 7-fold. Incubation of HalA1 with HalM2 or HalA2 with HalM1 did not result in modified peptide in either case, indicating that each HalM enzyme can dehydrate one but not both HalA peptides. These data also rule out the possibility that a complex involving both peptides and/or both proteins was required for activity of either enzyme. The activity of HalM1 with HalA1 was examined in the presence of HalA2 as well, and it was found that addition of the non-substrate peptide did not appear to inhibit modification of the true peptide substrate. Similar results were observed for HalM2 with HalA2 in the presence of HalA1.

### EXAMPLE 6

### Iodoacetamide Modification of the Haloduracin Peptides

The mass spectra recorded of the HalM assays demonstrated efficient dehydration activity but could not detect cyclization activity since no change in mass occurs in this step. To test for cyclization activity, the substrates and products were monitored for the presence of free cysteines by alkylation with iodoacetamide (IAA) following treatment with a reducing agent to assure cysteines would be available for reaction. Reaction with iodoacetamide results in the addition of a carbamidomethyl group to each free cysteine present, translating to an increase in mass of 58 Da. The authentic Hal1 and Hal2 peptides isolated from B. halodurans C-125 were first subjected to IAA modification. Hal1 displayed two adducts as judged by mass spectrometry (Δm=116 Da). Adduct formation was dependent on prior treatment with a reductant, indicating that the adducts formed on Cys residues that were tied up in a cystine linkage under non-reducing conditions. Hal2 did not undergo IAA adduct formation under any conditions tested, consistent with each Cys residue of this peptide being involved in a MeLan or Lan ring, which were not susceptible to chemical reducing agents.

To determine the importance of the disulfide in Hla1, the bioactivity of Hal1 and Hal2 after chemical modification with iodoacetamide was evaluated after removal of excess iodoacetamide using thiopropyl-SEPHAROSE resin. The remaining peptides containing two (Hal1) or zero (Hal2) adducts were then spotted against the indicator strain, where they exhibited zones of growth inhibition comparable to the wild-type peptides, indicating that the cystine linkage was not necessary for the biological activity of haloduracin.

To compare the ring structures of the haloduracin products produced in vitro with the peptides isolated from the producing strain, the products of the HalM assays were also treated with iodoacetamide and subjected to MALDI-TOF MS. The mass of HalA1 after modification by HalM1 was increased by 116 Da, consistent with the addition of two adducts. The mass of HalA2 modified by HalM2 remained unchanged under the alkylation conditions tested, consistent with the absence of free Cys residues. These results are in agreement with the chemical modification of the wild-type peptides, indicating that the HalM enzymes carried out the in vitro cyclization reaction in the same manner as in vivo.

### EXAMPLE 7

### Tests for the Presence of a 2-Oxobutyryl Group

The HalA2 peptide shared sequence homology with the β-peptide of plantaricin W (PlwAβ) and the LtnA2 peptide of lacticin 3147. Both of the mature peptides of these lantibiotics are thought to contain a 2-oxobutyryl group at their N-terminus (Martin, et al. (2004) Biochemistry 43:3049-3056; Holo, et al. (2001) Microbiology 147:643-651), resulting from spontaneous hydrolysis of an N-terminal Dhb. The position of removal of the leader peptide deduced from the mass of Hal2 isolated from B. halodurans, as well as its sequence, indicated Hal2 might also contain a 2-oxobutyryl residue at its N-terminus at position 42 of the prepeptide. To investigate this possibility, the peptide was first reacted with 1,2-diaminobenzene in a sodium acetate buffer to remove the oxobutyryl group (Martin, et al. (2004) supra; Stevens & Dixon (1995) Biochim. Biophys. Acta 1252:195-202; Sunde, et al. (1998) Biochim. Biophys. Acta 1388:45-52). Analysis by MALDI-TOF, however, did not show any change in the mass of the Hal2 peptide. A control reaction with a synthetic peptide 2-oxobutyryl-Ala-Trp-Pro-Ser (SEQ ID NO:40) showed the expected reaction under identical conditions, indicating that Hal2 did not contain an α-keto amide. In another experiment, rather than removing the 2-oxobutyryl group, the peptide was reacted with benzoyl hydrazine, which should result in a hydrazone adduct if a ketone group were present. However, no adduct was obtained with Hal2, whereas the control peptide showed the expected increase in mass due to hydrazone formation 8 (Δm=118 Da). Furthermore, the Hal1 and Hal2 peptides were analyzed by high resolution Fourier Transform mass spectrometry (, et al. (2006) J. Am. Chem. Soc. 128:1420-1), which can readily distinguish between peptides of different molecular formula. The masses of HalA1 and HalA2 were 3043.2802 Da (calculated 3043.2730 Da) and 2330.0456 Da (calculated 2330.0469 Da), respectively. The mass of Hal1 was within 2.5 ppm to a product with three dehydrations and one disulfide. The mass of Hal2 was consistent to 0.56 ppm with seven dehydrations and was inconsistent with an N-terminal 2-oxobutyryl group (calculated 2331.0309). Furthermore, analysis of the Hal2 peptide by tandem FTMS/MS resulted in fragment b-ions that clearly showed that in Hal2 Thr1, Thr2, Ser7, and Thr11 were dehydrated (corresponding to Thr42, Thr43, Ser48, and Thr52 in the HalA2 prepeptide) and that the one Ser/Thr residue that was not dehydrated was amongst Thr17, Thr 18, Thr 21 and Ser 22.

### EXAMPLE 8

### Engineering a Factor Xa Cleavage Site into the HalA Peptides

The biological activity of lantibiotic peptides is dependent upon the formation of the correct ring structures (Kuipers, et al. (1996) Antonievan Leeuwenhoek 69:161-169; Bierbaum, et al. (1994) Appl. Environ. Microbiol. 60:4332-8; Chen, et al. (1998) Appl. Environ. Microbiol. 64:2335-40; Ottenwalder, et al. (1995) Appl. Environ. Microbiol. 61:3894-903) and the removal of the N-terminal leader sequence of the modified peptides (van der Meer, et al. (1994) J. Biol. Chem. 269:3555-62; Xie, et al. (2004) Science 303:679-81). To demonstrate the biological activity of the haloduracin peptides prepared in vitro, the leader sequence of each product had to be removed. A peptide engineering method was used to achieve this goal. The last four amino acid residues of each peptide N-terminal to the cleavage site (as deduced from authentic Hal1 and Hal2) were replaced with the Factor Xa recognition sequence. In the case of HalA1, residues 38-41 (Val-Asn-Gly-Ala; SEQ ID NO:36) were replaced with the sequence Ile-Glu-Gly-Arg (SEQ ID NO:9) using molecular biology methods, whereas for HalA2 residues 32-25 (Val-His-Ala-Gln; SEQ ID NO:37) were replaced by Ile-Glu-Gly-Arg (SEQ ID NO:9). Because Factor Xa cleaves after the sequence Ile-Glu-Gly-Arg (SEQ ID NO:9), the structural region of each peptide obtained after digestion would correspond to the native mature products. The HalA peptides containing the Factor Xa cleavage site were overexpressed as hexahistidine fusion proteins and purified as described for the wild-type peptides. HalA1-Xa (HalA1 containing the Ile-Glu-Gly-Arg (SEQ ID NO:9) cleavage site) was incubated with HalM1 under the standard assay conditions to generate a 3-fold dehydrated species as judged by MALDI-TOF MS. HalA2-Xa (HalA2 containing the engineered cleavage site) was incubated with HalM2 under the same conditions to generate a 7-fold dehydrated species as judged by MALDI-TOF 9 MS. The results were consistent in both cases with the wild-type peptide data, indicating that substitution of four residues in the leader sequence of each peptide with the sequence Ile-Glu-Gly-Arg (SEQ ID NO:9) did not alter the recognition and activity of the HalM enzymes. Following HalM modification, each peptide was subjected to proteolysis by Factor Xa in a CaCl₂-dependent reaction. Application of the proteolyzed samples to the haloduracin sensitive strain L. lactis CNRZ 117 resulted in a zone of inhibition comparable to that produced by Hall and Hal2 isolated from B. halodurans. This zone was dependent on the addition of both modified peptides. When either peptide was spotted separately, no inhibition was observed.

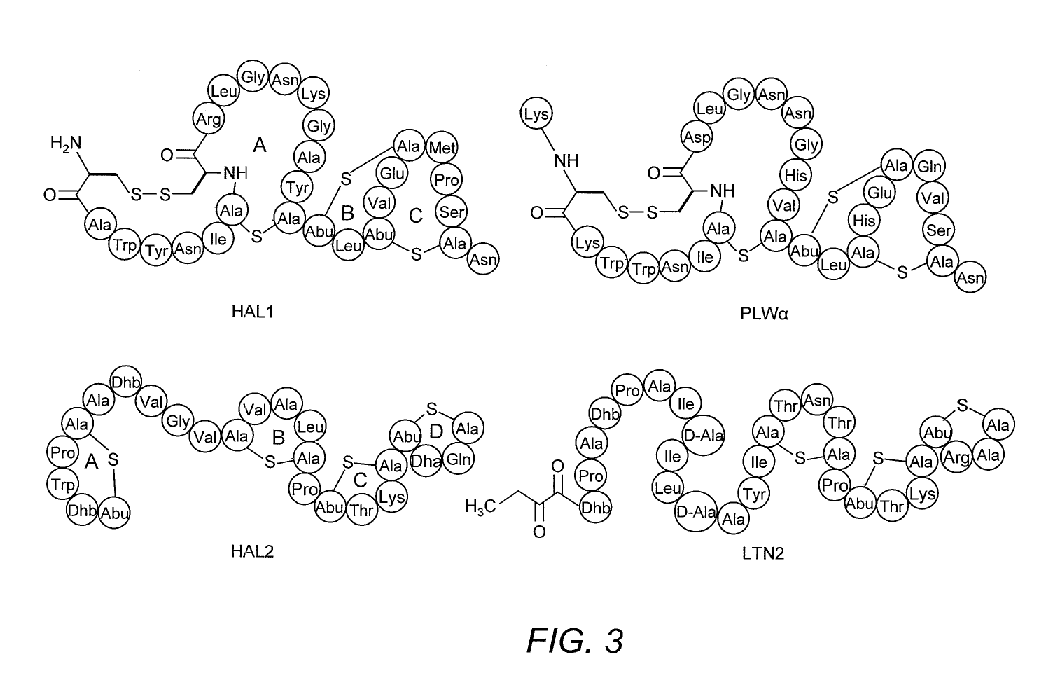

FIG. 3 depicts the structures for the two fully-processed haloduracin peptides that are consistent with mass spectrometric and structural characterization data presented herein, and with structural precedence in peptides from other systems. Based on the high accuracy mass spectrum of Hal1 isolated from the producing strain, HalA1 undergoes 3 dehydration events and the N-terminal leader sequence is removed after the anticipated proteolytic cleavage sequence Gly-Ala. The resulting product retains one Ser residue that was assigned to position 67, on the basis of similarity with the a peptide from plantaricin W (Plwα), which also contains an unmodified Ser residue at the equivalent position (Holo, et al. (2001) supra). The HalA1 structural peptide contains more Cys residues than Ser/Thr and hence not all cysteines can be engaged in Lan/MeLan rings. The formation of two IAA adducts only after pretreatment with reductants indicates that two cysteines are present in a cystine linkage in the isolated peptide. Cys42 and Cys49 are assigned to be involved based on similarity to Plwα. Hal1 is only the third example of a lantibiotic in which Cys residues are present as a disulfide, with sublancin and Plwα being the other examples (Holo, et al. (2001) supra; Paik, et al. (1998) J. Biol. Chem. 273:23134-42). As with plantaricin W (Holo, et al. (2001) supra), the oxidation state of these two Cys residues does not seem to be crucial for biological activity, since reduction and even alkylation with IAA did not abolish antimicrobial activity. The three remaining cysteines are believed to form one Lan and two MeLan rings with the same connectivity as confirmed (Martin, et al. (2004) supra) or proposed for all other known two-component lantibiotics (Holo, et al. (2001) supra; Navaratna, et al. (1998) Appl. Environ. Microbiol. 64:4803-8; Yonezawa & Kuramitsu (2005) supra; Hyink, et al. (2005) supra). The closest homolog, the a-peptide of plantaricin W, is shown in FIG. 3. The six-amino acid containing MeLan B-ring, which is believed to be important for lipid II binding in mersacidin (Hsu, et al. (2003) J. Biol. Chem. 278:13110-7), is conserved in HalA1 and LicA1 including the invariant and essential Glu within this ring (Szekat, et al. (2003) Appl. Environ. Microbiol. 69:3777-83). This MeLan ring is also found in the α/A1-peptides of lacticin 3147 (Martin, et al. (2004) supra), plantaricin W (Holo, et al. (2001) supra), staphylococcin C55 (Navaratna, et al. (1998) supra), Smb (Yonezawa & Kuramitsu (2005) supra), and BHT-A (Hyink, et al. (2005) supra) as well as in the lacticin 481 subgroup of single component lantibiotics (Chatterjee, et al. (2005) supra).

The accurate mass for Hal2 isolated from B. halodurans C-125 is consistent with 7-fold dehydration of the HalA2 prepeptide and cleavage of the leader peptide C-terminal to Gln41. As for Hall, the mass data indicate that Hal2 contains one unmodified Ser/Thr residue, assigned to Thr18 (Thr59 in HalA2) based on structural similarity with Ltn2 of lacticin 3147 (FIG. 3), as well as the FTMS/MS data that show the unmodified residue to be located in the segment spanning residues 16-24. Proteolytic processing after Gln41 of HalA2 would result in Dhb42 of HalA2 occupying the N-terminal position of Hal2 upon removal of the leader peptide. Eneamines are unstable in aqueous solutions and undergo spontaneous and rapid hydrolysis to the corresponding ketone, resulting in a 2-oxobutyryl residue instead of a Dhb (Kellner, et al. (1989) Angew. Chem. 101:618-21) (see Ltn2 in FIG. 3). Alternatively, if HalM2 catalyzes the formation of an N-terminal MeLan by reaction of Cys46 with Dhb42 of the prepeptide (Dhb1 and Cys5 in mature Hal2), Hal2 would not have an N-terminal Dhb upon proteolysis and hence no a-keto amide would be formed. The IAA alkylation experiments clearly showed that Cys5 of Hal2 was indeed involved in a MeLan since no free Cys was present. Furthermore, three independent methods provided evidence against an N-terminal 2-oxobutyryl group, indicating that HalM2 indeed forms a MeLan between residues 42 and 46 of HalA2. An alternative possibility that would result in the absence of the 2-oxobutyryl group is that Thr42 is not dehydrated resulting in Thr1 at the N-terminus of Hal2 after proteolysis. However, this model is inconsistent with the MS/MS data since the masses of a series of fragment ions clearly indicate the dehydration of Thr1 in Hal2. Unlike Cys5, the remaining three cysteines in Hal2 are conserved in the β/A2 peptides of lacticin 3147, plantaricin W, and staphylococcin C55, and hence their involvement in the Lan and MeLan rings shown in FIG. 3 is supported. Indeed the fragment ions observed are fully consistent with the proposed rings of Hal2, as is the lack of fragmentation in the segments spanning residues 1 and 5, 11 and 15, and 16 to 24 (Xie, et al. (2004) supra).

The mass data demonstrated that the cleavage site for the leader peptide is not at the predicted position as the LanT protease domains typically process their substrates at a double Gly recognition motif; for HalA2 this would have been between Ser35 and Gly36. A similar observation has been reported for the two-component systems plantaricin W and cytolysin, in which the peptide undergoes additional proteolytic processing beyond the removal of the leader sequence (Cox, et al. (2005) supra; Holo, et al. (2001) supra). In cytolysin the additional proteolysis has been shown to be necessary for biological activity (Cox, et al. (2005) supra)

What is claimed is:

1. An isolated two-component Bacillus lantibiotic comprising the amino acid sequences set forth in SEQ ID NO:1 and SEQ ID NO:2.

2. A pharmaceutical composition comprising the two-component Bacillus lantibiotic of claim 1 in admixture with a pharmaceutically acceptable carrier.

3. An isolated nucleic acid molecule encoding a Bacillus lantibiotic peptide comprising the amino acid sequence set forth in SEQ ID NO:1 or SEQ ID NO:2.

4. A vector comprising at least one of the nucleic acid molecules of claim 3.

5. A host cell comprising the vector of claim 4.

6. A method for producing the two-component Bacillus lantibiotic of claim 1, comprising contacting precursor peptides comprising SEQ ID NO:1 and SEQ ID NO:2 with at least one modifying enzyme capable of effecting dehydration and cyclization of the precursor peptide, and cleaving the leader peptide from the precursor peptides thereby producing a biologically active two-component Bacillus lantibiotic.

7. A method for modifying a peptide or polypeptide comprising contacting a peptide or polypeptide with a Bacillus lantibiotic modifying enzyme thereby effecting dehydration and cyclization of the peptide or polypeptide.

8. An isolated Bacillus lantibiotic modifying enzyme which effects dehydration and cyclization of a peptide or polypeptide.

9. An isolated nucleic acid molecule encoding the Bacillus lantibiotic modifying enzyme of claim 8.

10. A vector comprising the nucleic acid molecule of claim 9.

11. A host cell comprising the vector of claim 10.

12. A kit for producing haloduracin comprising HalA1, HalA2, HalM1 and HalM2.

13. A method for preventing or inhibiting the growth of a bacterium comprising contacting a bacterium with an effective amount of the two-component Bacillus lantibiotic of claim 1 thereby preventing or inhibiting the growth of the bacterium.

14. A method for preventing or treating a bacterial infection comprising administering to a subject in need of treatment an effective amount of the two-component Bacillus lantibiotic of claim 1 thereby preventing or treating a bacterial infection of the subject.
